(19) United States
(12) Patent Application Publication
(10) Pub. No.: US 2026/0240940 A1
(43) Pub. Date: Aug. 20, 2026

(54) REGULATING DISEASE OR DISORDER CHARACTERIZED BY ESOPHAGEAL EPITHELIAL PROLIFERATION THROUGH SECRETED FRIZZLED-RELATED PROTEIN 1 (SFRP1)

(71) Applicant: The Regents of the University of Michigan, Ann Arbor, MI (US)

(72) Inventors: Simon P. HOGAN, Ann Arbor, MI (US); Sahiti Marella, Ann Arbor, MI (US)

(21) Appl. No.: 19/472,356

(22) PCT Filed: Apr. 17, 2024
(86) PCT No.: PCT/US2024/025040

§ 371 Date: Oct. 3, 2025

Related U.S. Application Data
(60) Provisional application No. 63/459,847, filed on Apr. 17, 2023.

(51) Int. Cl. A61K 38/08; A61K 31/166; A61K 31/18; A61K 31/194; A61K 31/223; A61K 31/255; A61K 31/357; A61K 31/4402; A61K 31/4709; A61K 31/4725; A61K 31/551; A61K 31/555; A61K 31/58; A61K 31/661; A61K 31/7088; A61P 1/00

(52) U.S. Cl. CPC A61K 38/08; A61K 31/166; A61K 31/18; A61K 31/194; A61K 31/223; A61K 31/255; A61K 31/357; A61K 31/4402; A61K 31/4709; A61K 31/4725; A61K 31/551; A61K 31/555; A61K 31/58; A61K 31/661; A61K 31/7088; A61P 1/00

(57) ABSTRACT

The present disclosure is directed to compositions, methods, and agents directed to treating diseases and disorders characterized by abnormal esophageal epithelial proliferation and/or growth. In some embodiments, the compositions, methods, and agents comprise a SFRP1 activator or a composition thereof and/or SFRP1, a biologically active fragment or variant thereof, or a nucleic acid encoding SFRP1 or a biologically active fragment or variant thereof. In some embodiments, the compositions, methods, and agents comprise a signal transducer and activator of transcription 3 (STAT3) inhibitor or a composition thereof.

### STATEMENT REGARDING FEDERALLY-SPONSORED RESEARCH

This invention was made with government support under AI140133 awarded by the National Institutes of Health. The government has certain rights in the invention.

### FIELD

Disclosed herein are compositions, methods, and agents directed to treating diseases and disorders characterized by abnormal esophageal epithelial proliferation and/or growth with secreted frizzled-related protein 1 (SFRP1) and/or inhibition of signal transducer and activator of transcription 3 (STAT3).

### SEQUENCE LISTING STATEMENT

The contents of the electronic sequence listing titled UM-41736-601-ST26.xml (Size: 17,666 bytes; and Date of Creation: Apr. 17, 2024) is herein incorporated by reference in its entirety.

### BACKGROUND

Eosinophilic esophagitis (EoE) is histologically characterized by esophageal cosinophilia and epithelial remodeling, including basal zone hyperplasia (BZH) and dilated intercellular spaces (DIS). Esophageal epithelial remodeling is thought to contribute to esophageal barrier dysfunction and clinical manifestations of EoE; however, the molecular pathways that drive esophageal epithelial remodeling remain largely unexplored.

### SUMMARY

Provided herein are compositions, methods, and agents directed to treating diseases and disorders characterized by abnormal esophageal epithelial proliferation and/or growth. In some embodiments, the compositions, methods, and agents comprise a SFRP1 activator or a composition thereof and/or SFRP1, a biologically active fragment or variant thereof, or a nucleic acid encoding SFRP1 or a biologically active fragment or variant thereof. In some embodiments, the compositions, methods, and agents comprise a signal transducer and activator of transcription 3 (STAT3) inhibitor or a composition thereof.

In some embodiments, the disclosure provides a method of attenuating an esophageal epithelial proliferation disease in a subject in need thereof comprising increasing the level and/or the activity of secreted frizzled-related protein 1 (SFRP1) in the subject by administering to the subject an effective amount of a SFRP1 activator or a composition thereof. In some embodiments, the disclosure provides a method of attenuating an esophageal epithelial proliferation disease in a subject in need thereof comprising increasing the level and/or the activity of secreted frizzled-related protein 1 (SFRP1) in the subject by administering to the subject SFRP1, a biologically active fragment or variant thereof, or a nucleic acid encoding SFRP1 or a biologically active fragment or variant thereof. In some embodiments, the activator comprises a small molecule activator of SFRP1. The disclosure is not limited by the type of activator of SFRP1. In some cases, the activator comprises signal transducer and activator of transcription 3 (STAT3). In some embodiments, the SFRP1 comprises an amino acid sequence having at least 70% identity (e.g., 70%, 75%, 80%, 85%, 90%, 95%, 96%, 97%, 98%, 99% or higher identity, or a percent identity between and encompassing 70-100%) to SEQ ID NO: 13. In some embodiments, the SFRP1 comprises the amino acid sequence of SEQ ID NO: 13. A variety of esophageal epithelial proliferation diseases may be treated (e.g., attenuated) utilizing the compositions, methods and/or agents described herein. Examples of esophageal epithelial proliferation diseases that benefit from the compositions, methods and/or agents include, but are not limited to, an esophageal epithelial remodeling disorder, Basal Zone Hyperplasia (BZH) and Eosinophilic Esophagitis (EoE).

In some embodiments, the disclosure provides a method of treating or preventing an epithelial cell disease or disorder in a subject comprising administering to the subject a therapeutically or prophylactically effective amount of a signal transducer and activator of transcription 3 (STAT3) inhibitor or a composition thereof to the subject. Examples of epithelial cell diseases or disorders that benefit from the STAT3 inhibitor compositions, methods and/or agents include, but are not limited to, an esophageal epithelial remodeling disorder and/or esophageal epithelial proliferation disease (e.g., Basal Zone Hyperplasia (BZH) or Eosinophilic Esophagitis (EoE)). In some embodiments, the inhibitor is selected from a protein configured to bind STAT3 or a substrate thereof, a gene silencing oligonucleotide, a small molecule inhibitor of STAT3, a negative allosteric modulating agent, a protease, and combinations thereof. In some embodiments, the STAT3 inhibitor is peptide inhibitor. Examples of STAT3 peptide inhibitors include, but are not limited to, DBD-1, ISS-610, and PY*LKTK. In some embodiments, the STAT3 inhibitor is a peptide small molecule inhibitor. Examples of STAT3 small molecule inhibitors include, but are not limited to, PLLL32, HJC0152, LL1, LLL-3, LLL12, LYW-6, nitidine chloride, SD-36, static, STX-0119, S31-1757, S31-201, CPA-7, C48, GPA512, and MMPP. In some embodiments, the STAT3 inhibitor is an oligonucleodite (e.g., InS3-54A18 or STAT3hpdODN).

In some aspects, the disclosure provides a method of treating or preventing a disease or disorder in a subject comprising administering to the subject a therapeutically or prophylactically effective amount of at least one signal transducer and activator of transcription 3 (STAT3) inhibitor or a composition thereof. In some embodiments, the disease or disorder is characterized by aberrant esophageal epithelial proliferation. In some embodiments, the aberrant epithelial proliferation occurs within a suprabasal epithelial cell population of cells (e.g., characterized by markers identified in FIG. 8E (e.g., expression of cell surface proteins CD44, CD9 and HLAB) and/or expression of one or more inflammatory genes selected from TNFAIP6, POSTN, CCL26, and ALOX15). In some embodiments, the administration of the at least one STAT3 inhibitor attenuates aberrant esophageal epithelial proliferation, decreases epithelial cell remodeling, reduces inflammation, or a combination thereof. A variety of diseases or conditions may be treated (e.g., attenuated) utilizing the compositions, methods and/or agents described herein. Examples of aberrant esophageal epithelial proliferation that benefit from the compositions, methods and/or agents include, but are not limited to, an esophageal epithelial remodeling disorder, Basal Zone Hyperplasia (BZH) and Eosinophilic Esophagitis (EoE). In some embodiments, the STAT3 inhibitor is selected from a protein configured to bind STAT3 or a substrate thereof, a gene silencing oligonucleotide, a small molecule inhibitor of STAT3, a negative allosteric modulating agent, a protease, and combinations thereof. In some embodiments, the STAT3 inhibitor is peptide inhibitor. Examples of STAT3 peptide inhibitors include, but are not limited to, DBD-1, ISS-610, and PY*LKTK. In some embodiments, the STAT3 inhibitor is a peptide small molecule inhibitor. Examples of STAT3 small molecule inhibitors include, but are not limited to, PLLL32, HJC0152, LL1, LLL-3, LLL12, LYW-6, nitidine chloride, SD-36, static, STX-0119, S31-1757, S31-201, CPA-7, C48, GPA512, and MMPP. In some embodiments, the STAT3 inhibitor is an oligodeoxynucleotides (e.g., InS3-54A18 or STAT3 hairpin decoy oligodeoxynucleotides (hpdODN). In some embodiments, the method comprises administration of at least one additional therapeutic agent. A variety of therapeutic agents may be administered with a STAT3 inhibitor including, but not limited to, a chemotherapeutic, a corticosteroid, an immunosuppressant, an anti-inflammatory agent, an antibiotic, or any combination thereof.

The compositions, methods and systems of the disclosure are not limited by the route of administration of a therapeutically or prophylactically effective amount of at least one signal transducer and activator of transcription 3 (STAT3) inhibitor or a composition thereof, or a SFRP1 activator or a composition thereof and/or SFRP1, a biologically active fragment or variant thereof, or a nucleic acid encoding SFRP1 or a biologically active fragment or variant thereof. Indeed, any of one or more routes of administration may be used including, but not limited to, intravenously, arterially, intradermally, intramuscularly, intraperitoneally, subcutaneously, ocularly, sublingually, orally, intranasally, intraspinally, intracerebrally, and transdermally. In some embodiments, the disclosure provides a method of treating or preventing a disease or disorder in a subject, comprising administering to the subject an effective amount of secreted frizzled-related protein 1 (SFRP1), a biologically active fragment or variant thereof, or a nucleic acid encoding SFRP1 or a biologically active fragment or variant thereof. In some embodiments, the disease or disorder is characterized by aberrant esophageal epithelial proliferation. In some embodiments, the aberrant epithelial proliferation occurs within a suprabasal epithelial cell population of cells (e.g., characterized by markers identified in FIG. 8E (e.g., expression of cell surface proteins CD44, CD9 and HLAB) and/or expression of one or more inflammatory genes selected from TNFAIP6, POSTN, CCL26, and ALOX15). In some embodiments, the disclosure provides that SFRP1+ esophageal epithelial cells are a suprabasal epithelial cell population (e.g., that can be characterized and/or identified by markers described in FIG. 8E (e.g., expression of cell surface proteins CD44, CD9 and HLAB, and/or expression of one or more inflammatory genes such as TNFAIP6, POSTN, CCL26, and ALOX15) that underlie (e.g., cause and/or drive) esophageal epithelial disease (e.g., that can be targeted using one or more of the above described markers and/or targeted for treatment using one or more expressed cell surface proteins).

In some embodiments, the disease or disorder is an esophageal epithelial remodeling disorder. In some embodiments, the disease or disorder is Basal Zone Hyperplasia (BZH). In other embodiments, the disease or disorder is Eosinophilic Esophagitis (EoE). In some embodiments, the method of treating or preventing a disease or disorder comprises increasing the level and/or the activity of secreted frizzled-related protein 1 (SFRP1) in the subject by administering to the subject SFRP1, a biologically active fragment or variant thereof, or a nucleic acid encoding SFRP1 or a biologically active fragment or variant thereof. In some embodiments, the activator comprises a small molecule activator of SFRP1. The disclosure is not limited by the type of activator of SFRP1. In some cases, the activator comprises signal transducer and activator of transcription 3 (STAT3). In some embodiments, the SFRP1 comprises an amino acid sequence having at least 70% identity (e.g., 70%, 75%, 80%, 85%, 90%, 95%, 96%, 97%, 98%, 99% or higher identity, or a percent identity between and encompassing 70-100%) to SEQ ID NO: 13. In some embodiments, the SFRP1 comprises the amino acid sequence of SEQ ID NO: 13. In some embodiments, the method comprises also administering a signal transducer and activator of transcription 3 (STAT3) inhibitor or a composition thereof.

In some aspects, the disclosure provides use of a SFRP1 activator or a composition thereof; and/or SFRP1, a biologically active fragment or variant thereof, or a nucleic acid encoding SFRP1 or a biologically active fragment or variant thereof for use in treating a disease or disorder characterized by esophageal epithelial proliferation.

In other aspects, the disclosure provides use of a SFRP1 activator or a composition thereof; and/or SFRP1, a biologically active fragment or variant thereof, or a nucleic acid encoding SFRP1 or a biologically active fragment or variant thereof for use in treating a disease or disorder characterized by esophageal epithelial remodeling.

In some embodiments, the disclosure provides use of a SFRP1 activator or a composition thereof; and/or SFRP1, a biologically active fragment or variant thereof, or a nucleic acid encoding SFRP1 or a biologically active fragment or variant thereof for use in regulating esophageal epithelial proliferation.

Other aspects and embodiments of the disclosure will be apparent in light of the following detailed description and accompanying figures.

### BRIEF DESCRIPTION OF THE DRAWINGS

FIGS. 1A-1D shows enrichment of STAT3-regulated genes in EoE and IL-13-stimulated EPC2-ALI. RNA sequencing (RNAseq) datasets and associated differentially expressed gene (DEG) counts (FIG. 1A). Venn diagram of common and unique DEGs from three independent RNAseq datasets (FIG. 1B) UpSet plot of putative STAT protein targets from the 82 common esophageal epithelial-specific EoE genes (FIG. 1C) List of putative STAT3 targets from the 82 esophageal epithelial-specific EoE genes (FIG. 1D). NL represent healthy control patients.

FIGS. 2A-2K show IL-13 activates STAT3 in EPC2-ALI and primary esophageal cells derived from patient biopsies. Schematic of EPC2-ALI culture system (FIG. 2A). Western blot of EPC2-ALI under Vehicle and IL-13-stimulated conditions with probing for phosphorylation of STAT3 (Y705, S727) and STAT6 (Y641) (FIG. 2B). Schematic of STAT3 and STAT6 activation kinetics from EPC2-ALI stimulated with Vehicle or IL-13 time-course (FIG. 2C). Representative diagram of 3D esophageal epithelial cell organization and marker expression with Krt15-expressing basal cells at the basolateral surface and Krt4-expressing squamous cells towards the apical surface (FIG. 2D). Immunofluorescence (IF) staining of 3D primary esophageal epithelium stained for Krt15 (green), Krt4 (red), and DAPI (blue) (FIG. 2E). Immunofluorescence image from the basolateral surface of the 3D stack with Krt15+ basal epithelial cells (FIG. 2F). Immunofluorescence image from the apical surface of the 3D stack with Krt4+ squamous epithelial cells (FIG. 2G). Immunofluorescence staining of basal cells (Krt15; green) and proliferative cells (Ki-67; pink). (FIG. 2H). Western blot of IL-13-stimulated primary esophageal cells for STAT3 and STAT6 phosphorylation (FIG. 2I). qPCR mRNA fold-change of ANO1 and CCL26 in IL-13-stimulated primary esophageal cells with representative passages 3 and 6 (FIG. 2J and FIG. 2K, respectively). Repeated-measures ANOVA was performed on IL-13 time-course Western blot densitometry analyses to determine the statistical significance of IL-13 stimulation on expression of proteins over time. Ordinary 1-way ANOVAs with the Tukey multiple comparisons test were performed on qPCR analyses of the mean fold-change expression values across groups. *P<0.05; **P<0.01; ***P<0.001; ****P<0.0001.

FIGS. 3A-3L show that mice with inducible overexpression of IL-13 in esophageal epithelial cells exhibit epithelial remodeling and increased STAT3 expression. Schematic of in vivo model of IL-13-induced esophageal epithelial remodeling (FIG. 3A). H&E staining of Dox1 Krt5-rtTA×WT (WT) and Dox1 Krt5-rtTA×TetO-IL-13Tg (Tg) mouse esophagus (yellow arrows: DIS; green bars: BZH; red bars: total epithelial thickness) (FIGS. 3B and 3C, respectively). Immunohistochemistry staining for Ki67 for esophageal basal proliferation of WT and Tg esophagus (green arrows: Ki671 cells) (FIGS. 3D and 3E, respectively). Masson trichrome staining for collagen deposition in WT and Tg mice (FIGS. 3F and 3G, respectively). Measurement of total epithelial thickness (FIG. 3H). Percentage of basal zone to total epithelial thickness (FIG. 3I). Measurement of DISs (nM) (FIG. 3J). Quantification of Ki671 cells/mm epithelium (FIG. 3K). Spearman rank correlation analysis between Ki671 cells/mm epithelium and esophageal epithelial thickness in WT and Tg mice (FIG. 3L). Individual data points represent 1 mouse. Welch t tests were performed to compare differences in quantifications of epithelial thickness, percent basal zone, DISs, and Ki671 cells/mm epithelium between WT and Tg mice. *P<0.05; **P<0.01; ***P<0.001; ****P<0.0001. Data are represented as means 6 SEMs of 3 independent experiments.

FIGS. 4A-4E show STAT3 protein expression in mice with inducible overexpression of IL-13 in esophageal epithelial cells. IHC staining of STAT3 protein in WT mouse esophagus with no primary antibody (FIG. 4A). IHC staining of STAT3 protein in WT mouse esophagus (FIG. 4B). IHC staining of STAT3 protein in Tg mouse esophagus (FIG. 4C). FIGS. 4D and 4E show immunohistochemistry staining of phosphorylated STAT3 (pSTAT3-Y705) in WT mouse esophagus (FIG. 4D) and Tg mouse esophagus (FIG. 4E). All tissues were counterstained with nuclear hematoxylin stain.

FIGS. 5A-5K show IL-13-induced esophageal epithelial proliferation requires STAT3. Immunofluorescence staining of Ki-67 (green) and DAPI of EPC2CTRL and EPC2ΔSTAT3 under Vehicle and IL-13 stimulation (FIG. 5A). Quantification of Brdu+ cells/mm membrane (FIG. 5B). Western blot of EPC2-ALI probing for total (TSTAT3) and phosphorylated STAT3 (pSTAT3-Y705 and pSTAT3-S727) in the presence and absence of IL-13 and selective STAT3 degrader, SD-36 (FIG. 5C). Depiction of epithelial remodeling (BZH, DIS) with Hematoxylin & Eosin staining in the presence and absence of IL-13 and SD-36; green arrow points to DIS and yellow brackets mark basal cell expansion (FIG. 5D). IF staining for Ki-67 (red) for basal cell proliferation KI67 of EPC2-ALI in the presence and absence of IL-13 and SD-36 (FIG. 5E). Quantification of Ki-67+ cells/mm membrane (FIG. 5F). qPCR for mRNA fold-change of ANO1, TP63, CCL26, SLC9A3, and CAPN14, respectively, in the presence and absence of IL-13 and SD-36 (FIG. 5G-5K, respectively). Ordinary 1-way ANOVA with the Tukey multiple comparisons test was performed on qPCR analyses and quantification of BrdU1 cells/mm membrane of the mean fold change expression values across groups. Data are represented as means 6 SEMs of 3 independent experiments (n 5 3 samples/group). NS, Not significant. *P<0.05; **P<0.01; ***P<0.001; ****P<0.0001.

FIGS. 6A-6E show SFRP1 is a novel candidate gene that is associated with cell proliferation and is a STAT3 target. Venn diagram of common and unique DEGs from three independent RNAseq datasets, showing 82 common esophageal epithelial-specific EoE genes (FIG. 6A). Network map of the esophageal epithelial-specific EoE DEGs identifying candidate gene, SFRP1 (gray circle: common DEGs from FIG. 1B; gray square: putative STAT3 targets; purple circle: associated with “Cell Proliferation” GO term; orange square: putative STAT3 target and associated with “Cell Proliferation” GO term; pink square: putative STAT3 target (FIG. 6B). qPCR mRNA fold-change of SFRP1 in EPC2-ALI cells stimulated with Vehicle and IL-13 at 4- and 48-hours. (FIG. 6C). qPCR mRNA fold-change of SFRP1 in EPC2-ALI cells stimulated with Vehicle and IL-13 in the presence and absence of SD-36 (FIG. 6D). Immunohistochemistry staining of SFRP1 protein in Krt5-rtTA×WT mouse esophagus (FIG. 6E) and Krt5-rtTA×TetO-IL-13Tg mouse esophagus (FIG. 6F). qPCR data are represented as means 6 SEMs of 3 independent experiments (n 5 3 samples/group). Staining was performed on mouse tissues extracted from 3 independent experiments. NL, Healthy control patients. *P<0.05; **P<0.01; ***P<0.001; ****P<0.0001.

FIGS. 7A-7H show esophageal epithelial cells are cellular sources of SFRP1 expression and IL-13-induced esophageal proliferation is regulated by SFRP1. Seurat-integrated scRNA-seq samples consisting of 5 active, 3 remission, and 2 normal esophageal patient biopsies (FIG. 7A). scRNA-seq clustering of integrated samples based on major cell types that are annotated as epithelial, mast, endothelial, fibroblast, monocyte, and lymphocyte (FIG. 7B). mRNA expression of SFRP1 among the 6 annotated clusters (FIG. 7C). Cell-type-specific mRNA expression of SFRP1 separated by disease state (active, normal, and remission) (FIGS. 7D, 7E, and 7F, respectively). Immunofluorescence staining of Ki67 and quantification in EPC2-ALI in the presence or absence of IL-13, rSFRP1, and SFRP1 pharmacologic antagonist, WAY316606 (SFRP1inh) (FIGS. 7G and 7H, respectively). Ordinary 1-way ANOVA with the Tukey multiple comparisons test was performed on quantification of Ki671 cells/mm membrane of the mean fold-change expression values across groups. Data are represented as means 6 SEMs of 3 independent experiments (n 5 3 samples/group). NS, Not significant; UMAP, Uniform Manifold Approximation and Projection. *P<0.05; **P<0.01; ***P<0.001; ****P<0.0001.

FIGS. 8A-81 show a novel esophageal epithelial cell cluster present exclusively in Active EoE scRNAseq samples is a potent source of SFRP1. Sub-clustering of the integrated and combined “Epithelial Cell” cluster identified in FIG. 7B into six predominant subpopulations (FIG. 8A). Subclustered epithelial populations separated by disease state (active, normal, and remission) and identification of a seventh subpopulation in the “Active” samples. (FIG. 8B). SFRP1 mRNA expression in epithelial subpopulations separated by disease state (active, normal, and remission) (FIG. 8C). Mapping of 560 genes expressed in the seventh epithelial subpopulation present in active disease, on the top 100 “Basal” and “Suprabasal” markers (FIG. 8D). Network of top 50 DEGs present in the disease-associated epithelial population, depicting canonical genes highly differentially expressed in EoE (green) (FIG. 8E). mRNA expression of EoE proinflammatory genes CCL26, ALOX15, TNFAIP6, and POSTN in the disease-associated epithelial population. UMAP, Uniform Manifold Approximation and Projection (FIGS. 8F, 8G, 8H, and 8I, respectively).

FIG. 9 shows full length, wild type amino acid sequence of human SFRP1.

### DETAILED DESCRIPTION

The present disclosure is directed to compositions, methods, and agents directed to treating diseases and disorders characterized by abnormal esophageal epithelial proliferation and/or growth. In some embodiments, the compositions, methods, and agents comprise a SFRP1 activator or a composition thereof and/or SFRP1, a biologically active fragment or variant thereof, or a nucleic acid encoding SFRP1 or a biologically active fragment or variant thereof. In some embodiments, the compositions, methods, and agents comprise a signal transducer and activator of transcription 3 (STAT3) inhibitor or a composition thereof.

Definitions. To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “treat,” “treating” and the like means a slowing, stopping, or reversing of progression of a disease or disorder or reducing the severity or activity thereof when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

As used herein, the term “preventing” refers to partially or indefinitely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

A “subject” or “patient” may be human or non-human and may include, for example, animal strains or species used as “model systems” for research purposes, such a mouse model. Likewise, patient may include either adults, juveniles (e.g., children), or infants. Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

1. Compositions, Methods, and Agents Directed to Treating Diseases and Disorders Characterized by Abnormal Esophageal Epithelial Proliferation and/or Growth

Eosinophilic Esophagitis (EoE) is a food antigen-mediated chronic inflammatory disease of the esophagus clinically characterized by upper gastrointestinal (GI) symptoms including, dysphagia, vomiting, and food impaction (1-6). The histopathology of EoE is defined by eosinophil-rich inflammation (15 eosinophils per high-power-field [Eos/HPF]) and esophageal remodeling, consisting of basal zone hyperplasia (BZH) and dilated intercellular spaces (DIS) (1, 2, 4, 5, 7-9). BZH and DIS is thought to contribute to esophageal barrier dysfunction, luminal narrowing, and chronic inflammation and the fibrostenotic phenotype in EoE (1, 6, 10-13).

Corroborative clinical and experimental studies support the concept that an underlying allergic sensitization to dietary food antigens and development of a CD4+Th2 and ILC2 inflammatory response in the esophageal mucosa drive the eosinophilic inflammation and esophageal remodeling in EoE (2,14-16). The frequency of CD4+Th2 cells in the peripheral blood and esophageal biopsy samples from EoE individuals (17-19) are increased and the percentage of CD4+Th2 cells correlated with esophageal tissue cosinophilia (19). Furthermore, scRNAseq analyses of esophageal biopsy samples from EoE patients revealed a prominent tissue resident CD4+ T cell population that expressed IL-4, IL-5 and IL-13 (19).

IL-13 is thought to be the central cytokine that drives the allergic inflammatory response and histopathological features of EoE. IL-13-induced esophageal epithelial transcriptomic changes as well as transcriptional changes observed in the esophagus of mice engineered to overexpress IL-13 significantly overlap with that of esophageal biopsy samples of EoE patients (20, 21). Treatment of adult EoE patients with RPC4046, a recombinant humanized monoclonal antibody against IL-13 improves histologic and endoscopic outcomes and patients that completed a 52-week open-label, long-term extension (LTE) study receiving open-label RPC4046 360 mg/week demonstrated sustained endoscopic, histologic and clinical improvement (22, 23). Furthermore, a phase 2 multi-center study of adults with active EoE who received weekly subcutaneous injections of dupilumab, a fully human IgG4 monoclonal antibody that binds the IL4Rα chain and blocks both IL-4 and IL-13 signaling, demonstrated reduced peak esophageal eosinophil count, decreased histologic and endoscopic severity scores, and increased esophageal distensibility (24, 25). IL-13 is thought to dysregulate the expression of several key epithelial barrier regulatory genes including desmosomal cadherin desmoglein-1 (DSG1), leucine-rich repeat-containing protein 31 (LRRC31), kallikrein (KLK) serine proteases and calpain-14 (CAPN14) and promote esophageal epithelial proliferation altering esophageal epithelial barrier function and inducing esophageal remodeling (7, 20, 26).

IL-13 can signal through the Type II IL-4 receptor that is composed of an IL-4Rα and IL-13Rα1 chain and the IL-13 receptor which consists of the IL-13Rα1 and IL-13Rα2 chains (27-32). IL-13 binding to the Type II IL-4 receptor leads to activation of downstream JAK1- or JAK2−/TYK2 and subsequent phosphorylation of STAT6 and STAT3. IL-13-induced phosphorylation of STAT6 induces expression of key EOE inflammatory genes including CCL26 and CAPN14 (33). Intratracheal IL-13 administration to wild-type mice resulted in dose-dependent esophageal eosinophilia and EoE-like pathophysiology, which was ablated in STAT6-deficient mice (34). On the contrary, the contribution of IL-13-induced STAT3 signaling to the inflammatory and esophageal epithelial remodeling response is not defined. STAT3 is phosphorylated at Y705 and S727 residues. Phosphorylation at the Y705 residue is required for STAT3 dimerization and nuclear translocation, and phosphorylation at the S727 residue promotes STAT3 mitochondrial translocation and enhances gene transcription (27-32, 35-38). Importantly, STAT3 phosphorylation and activation has been implicated in many cellular functions including cell growth, apoptosis, cell migration and proliferation.

As described herein (e.g., in the Examples), experiments conducted during development of embodiments of the disclosure identified 82 genes that were differentially expressed in EoE patient biopsies and IL-13-stimulated EPC2-ALI. Computational analysis revealed that these differentially expressed genes (DEGs) were enriched for putative STAT3 targets. Employing esophageal epithelial keratinocytes, a complex esophageal culture system derived from patient biopsies and an in vivo model of esophageal IL-13-overexpression, IL-13 was discovered to induce esophageal epithelial STAT3 phosphorylation and activation (STAT3Y705 and STAT3S727) and esophageal epithelial proliferation. Importantly, STAT3 was required for IL-13-induced esophageal epithelial proliferation and expression of EoE proliferative genes. In silico analyses identified the putative STAT3 target Secreted frizzled-related protein 1 (SFRP1) as a DEG associated with the IL-13-induced esophageal epithelial proliferative network. IL-13 induced SFRP1 mRNA expression in esophageal epithelial cells via a STAT3-dependent mechanism. Agonism and antagonism of SFRP1 revealed that SFRP1 plays a key regulatory role in IL-13-induced STAT3-dependent esophageal proliferation. Strikingly, SFRP1 was discovered to be expressed predominantly in esophageal epithelial cells in Active EoE compared to EoE in remission or Normal control samples and SFRP1+ esophageal epithelial cells were enriched for the core EoE pro-inflammatory transcriptome (e.g., CCL26, ALOX15, TNFAIP6, POSTN, ANO1). Thus, in some embodiments, the disclosure provides SFRP1 as a key regulator of IL-13-induced and STAT3-dependent esophageal proliferation and BZH in EoE, and identification of SFRP1+ esophageal epithelial cell population in esophageal inflammation and remodeling in EoE. In some embodiments, the disclosure provides compositions, methods, and agents directed to treating diseases and disorders characterized by abnormal esophageal epithelial proliferation and/or growth (e.g., compositions, methods, and agents comprising a SFRP1 activator or a composition thereof and/or SFRP1, a biologically active fragment or variant thereof, or a nucleic acid encoding SFRP1 or a biologically active fragment or variant thereof, or, compositions, methods, and agents comprise a signal transducer and activator of transcription 3 (STAT3) inhibitor or a composition thereof).

In some embodiments, the present disclosure provides (e.g., in Example 1) in vitro culture systems, a murine model, and in silico analyses of human datasets demonstrating a critical role for SFRP1 in IL-13-induced STAT3-dependent esophageal epithelial proliferation (e.g., in EoE). The disclosure further provides that 1) IL-13 induces STAT3 activation and STAT3-driven transcriptional programs in esophageal epithelial cells; 2) IL-13-induced proliferation is regulated by a STAT3-dependent mechanism; 3) that inducible IL-13 overexpression in Krt5⁺ esophageal cells increased total STAT3 protein expression and resulted in esophageal epithelial remodeling (e.g., epithelial thickness, BZH and DIS); 4) IL-13 induces SFRP1 mRNA expression via STAT3-dependent mechanism; 5) SFRP1 counter-regulates IL-13-induced esophageal epithelial proliferative response; 6) SFRP1 is expressed by esophageal suprabasal epithelial cells; and 7) SFRP1⁺ suprabasal epithelial cells are a subset of epithelial cells that present in active disease and express the core EoE pro-inflammatory and pro-proliferative transcriptional programs.

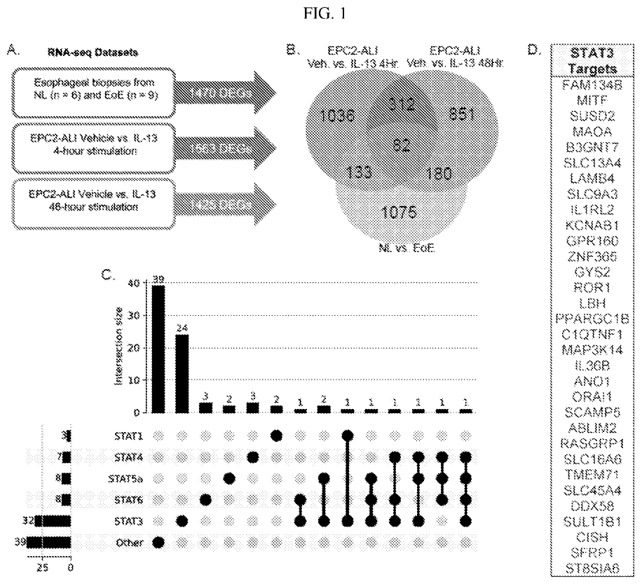

IL-13 is known to activate the transcription factors STAT3 and STAT6 in both hematopoietic and non-hematopoietic cell compartments (27, 48). A majority of these studies have focused on IL-13 induction of STAT6 and this pathway's role in driving Th2-inflammatory responses in allergic inflammatory diseases such as asthma, atopic dermatitis, and EoE (49). In EoE, the IL-13-STAT6 signaling axis is known to induce key inflammatory genes such as CCL26 and CAPN14 and is thought to drive the eosinophilic inflammatory response (33, 50). Consistent with this IL-13-induced histopathology and eosinophilic inflammation in the esophagus of mice is STAT6-dependent (51). The present disclosure provides in-silico bulk-RNAseq analyses that identified an esophageal epithelial-specific EoE transcriptome comprising n=82 DEGs that were predominantly putative STAT3 targets and were enriched for biological processes related to proliferation (e.g., 32 (39%) of which were putative STAT3 targets (See FIG. 1D). STAT3-induced transcriptional programs have been shown to be involved in homeostatic functions including tissue development, maturation as well as inflammation and immunity (52). STAT3 transcriptional activity is predominantly dependent on tyrosine (Y705) and serine (S727) phosphorylation and activation which promotes STAT3 dimerization and translocation to the nucleus where it induces transcriptional programs (52). STAT3 is considered an oncogene activating transcriptional programs that regulate multiple cellular processes such as proliferation, survival, and metastasis (53). For example, persistent STAT3 activation induces the expression of cell-cycle genes c-Myc, CyclinD1, and Survivin (53). Furthermore, STAT3 also signals to suppress apoptosis in cancer cells through upregulation of anti-apoptotic genes such as Bcl2, Bcl-XL, and Mcl1 (53). In some embodiments, the present disclosure provides that IL-13 stimulation of esophageal epithelial cells induces a time-dependent increase in both STAT3 and STAT6 phosphorylation. Lentiviral knockdown of STAT3, and not STAT6, in EPC2-ALI cells inhibited IL-13-induced esophageal epithelial proliferation, indicating that IL-13 induced esophageal epithelial proliferation was STAT3-dependent.

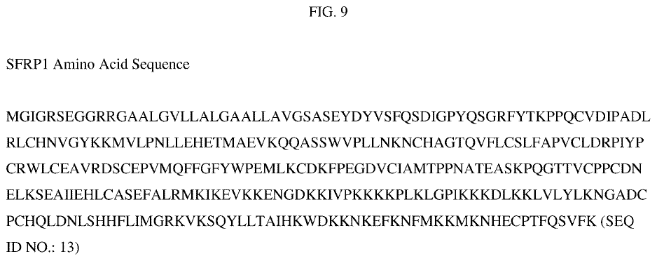

Experiments conducted during development of embodiments of the disclosure identified SFRP1, a putative STAT3 target and a soluble modulator of the Wnt signaling pathway as a common DEG. SFRP1 is a secreted glycoprotein that is member of the secreted frizzled-related receptor family (1-5) which act as soluble modulators of Wnt signaling. The amino acid sequence of human SFRP1 is shown in FIG. 9. In the canonical Wnt/β-catenin pathway, Wnt binds to Wnt receptor, Frizzled (Fzd) leading to inactivation of the β-catenin destruction complex and dissociation of β-catenin enabling b-catenin to traffic to the nucleus and induce transcription of target proliferation genes such as c-myc and Cyclin-D1 (CDK1) (45, 59-61). In the absence of Wnt, β-catenin is phosphorylated and bound to the destruction complex where it undergoes ubiquitin-mediated proteasomal degradation preventing transactivation of proliferative genes (45, 59-61). SFRP1 acts as competitive antagonist binding to Wnt ligand through the netrin (NTR) domain and preventing Wnt binding to its receptor, Fzd. SFRP1 can also indirectly inhibit Wnt by binding to cytoplasmic β-catenin and preventing downstream gene transcription or by binding to the Fzd receptor through the cysteine-rich (CRD) domain and preventing binding of Wnt ligands to the receptor. These three antagonistic mechanisms of SFRP1 promote downstream proteasomal degradation of β-catenin and negative regulation of β-catenin-induced pro-survival and pro-proliferation transcriptional programs. Accordingly, in some embodiments, compositions, methods, and systems disclosed herein are utilized to modulate proteasomal degradation of β-catenin and/or negative regulation of β-catenin-induced pro-survival and/or pro-proliferation transcriptional programs.

The disclosure provides that IL-13-induces a rapid increase in SFRP1 mRNA in esophageal epithelial cells via STAT3-dependent process. Furthermore, IL-13-induced proliferation in the presence of recombinant SFRP1 inhibited esophageal epithelial proliferation. In some embodiments, the disclosure provides compositions, methods, and systems that alter SFRP1 expression (e.g., SFRP1 expression in esophageal epithelial cells to regulate IL-13-induced esophageal epithelial proliferation).

The disclosure further provides, utilizing the murine Krt5-rtTA×tetO-IL-13Tg model, that IL-13 induction led to increased STAT3 within the epithelial compartment and that this was associated with esophageal epithelial proliferation and BZH. Accordingly, in some embodiments, IL-13-induction of STAT3 and the esophageal proliferative response contributes to esophageal epithelial remodeling such as BZH and DIS, and thus, the disclosure provides that modulation of STAT3 can be used to modulate (e.g., reduce) esophageal epithelial remodeling such as BZH and DIS.

The present disclosure also provides that the predominant cellular source of SFRP1 in the esophageal mucosa was a novel subtype of epithelial cells that were restricted to active disease, that express key canonical EoE inflammatory genes including CCL26 (66), TNFAIP6 (67), and ALOX15, which is expressed in esophageal biopsies from patients with definitive EoE (68). Thus, in some embodiments, the disclosure identifies a subset of suprabasal epithelial cells as the key cell population that promotes esophageal eosinophilia and the proinflammatory esophageal landscape. This subset of suprabasal epithelial cells also expressed canonical genes associated with epithelial remodeling including ANO1 and SLC9A3, which has been shown to have a role in IL-13-induced esophageal epithelial remodeling. Thus, the disclosure provides that a subset of suprabasal epithelial cells as the key disease driving entities which not only promote a pro-proliferative phenotype but also possess a strong pro-inflammatory transcriptional landscape. Identification of the IL-13-STAT3-SFRP1 signaling axis and its implications on BZH and EoE esophageal remodeling are novel. Accordingly, in some embodiments, compositions, methods, and/or systems of the disclosure are utilized to modulate epithelial remodeling disease (e.g., EoE) that is driven largely by this subset of suprabasal epithelial cells (e.g., by altering the pro-inflammatory gene signature and/or mediating the Th2 immune response which is characteristic of epithelial remodeling disease (e.g., EoE).

### 2. Methods of Treating Disease or Disorder

The disclosure provides methods of treating or preventing a disease or disorder in a subject comprising administering to the subject a therapeutically or prophylactically effective amount of an SFRP1 activator or a composition thereof and/or SFRP1, a biologically active fragment or variant thereof, or a nucleic acid encoding SFRP1 or a biologically active fragment or variant thereof, as described above, or a signal transducer and activator of transcription 3 (STAT3) inhibitor or a composition thereof, as described herein.

Compositions comprising an SFRP1 activator or a composition thereof and/or SFRP1, and/or STAT3 inhibitor may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, com starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. Techniques and formulations may be found, for example, in Remington's Pharmaceutical Sciences, 19th Edition (Mack Publishing Company, 1995). The route or administration and the form of the composition usually dictates the type of carrier to be used.

The compositions may be formulated for any appropriate manner of administration, and thus administered, including for example, oral, nasal, intraocular, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage.

The terms “effective amount” or “therapeutically effective amount,” as used herein, refer to a sufficient amount of a composition described herein being administered which will relieve to some extent one or more of the symptoms of the disease or condition being treated (e.g., abnormal esophageal epithelial proliferation and/or growth). The result can be reduction and/or alleviation of the signs, symptoms, or causes of a disease, or any other desired alteration of a biological system. For example, an “effective amount” for therapeutic uses is the amount of a compositions required to attenuate and/or modulate abnormal esophageal epithelial proliferation and/or growth.

The amount of the composition required for use in treatment or prevention will vary not only with the particular composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, in some embodiments, useful dosages of compositions required to attenuate and/or modulate abnormal esophageal epithelial proliferation and/or growth can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each composition disclosed herein but can be estimated from in vivo and/or in vitro data. Dosages necessary to achieve the MEC will depend on individual characteristics and route of administration. However, bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the composition may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Composition described herein can be evaluated for efficacy and toxicity using known methods. For example, the toxicology of a particular composition can be established by determining in vitro toxicity towards a cell line, such as a mammalian, and preferably human, cell line. The results of such studies are often predictive of toxicity in animals, such as mammals, or more specifically, humans. Alternatively, the toxicity of particular composition in an animal model, such as mice, rats, rabbits, dogs, or monkeys, may be determined using known methods. The efficacy of a particular composition may be established using several recognized methods, such as in vitro methods, animal models, or human clinical trials. When selecting a model to determine efficacy, the skilled artisan can be guided by the state of the art to choose an appropriate model, dose, route of administration and/or regime.

An effective amount of a composition of the disclosure may be administered alone or in combination with at least one additional therapeutic agent. In some embodiments, the at least one additional therapeutic agent is administered prior to, concomitantly with, or following administration of the composition (e.g., an SFRP1 activator or a composition thereof and/or SFRP1, a biologically active fragment or variant thereof, or a nucleic acid encoding SFRP1 or a biologically active fragment or variant thereof, as described herein, or a signal transducer and activator of transcription 3 (STAT3) inhibitor or a composition thereof, as described herein). The additional therapeutic agent may include, for example, a corticosteroid, an immunosuppressant, rituximab, anti-inflammatory agent, an antibiotic, an opioid antagonist, a vitamin or nutritional supplement, or any combination thereof. The choice of additional therapeutic agents will not only depend on the particular disease or disorder but also the nature and/or symptoms of the disease and the age and condition of the patient.

The disease or disorder may be characterized by abnormal esophageal epithelial proliferation and/or growth. As such, the disease or disorder may comprise an inflammatory disease, a fibrosis disease, infectious disease, and an autoimmune disease. In some embodiments, the disease or condition is a Th2-inflammatory related and/or allergic inflammatory disease (e.g., asthma, atopic dermatitis, or EoE).

In some embodiments, the disease or disorder is an inflammatory disease or disorder. Inflammatory diseases are characterized by activation of the immune system in a tissue or an organ to abnormal levels that may lead to abnormal function, dysfunction and/or disease in the tissue or organ.

Some autoimmune disorders are also associated with an inflammatory condition. Examples of inflammatory disorders which are also autoimmune disorders that can be prevented, treated, or managed in accordance with the methods of the disclosure include, but are not limited to, asthma, allergic disorders, and chronic inflammation resulting from chronic viral or bacterial infections.

Compositions (e.g., an SFRP1 activator or a composition thereof and/or SFRP1, a biologically active fragment or variant thereof, or a nucleic acid encoding SFRP1 or a biologically active fragment or variant thereof, as described herein, or a signal transducer and activator of transcription 3 (STAT3) inhibitor or a composition thereof) described herein may be administered to the subject by a variety of methods know to those skilled in the art, including without limitation, systemic administration (e.g., oral, rectal, nasal, sublingual, buccal, implants, or parenteral) or topical administration (e.g., dermal, pulmonary, nasal, aural, ocular, liposome delivery systems, or iontophoresis). In some embodiments, administration is directly into the site of disease, inflammation, or fibrosis. For example, in some embodiments, administration is directly into esophageal tissue.

As used herein, the terms “treatment”, “therapeutic use”, or “medicinal use” refer to any and all uses of the disclosed compositions (e.g., a SFRP1 activator or a composition thereof; SFRP1, a biologically active fragment or variant thereof, or a nucleic acid encoding SFRP1 or a biologically active fragment or variant thereof; STAT3 or inhibitor thereof) that remedy a disease state or symptoms, or otherwise prevent, hinder, retard, or reverse the progression of disease or other undesirable symptoms in any way whatsoever. For example, the terms “treatment of cancer” or “treatment of tumor” or “treatment of an esophageal epithelial proliferation disease” or grammatical equivalents herein are meant the suppression, regression, or partial or complete disappearance of a pre-existing cancer or tumor or esophageal epithelial proliferation disease. The definition is meant to include any diminution in the size, potency or growth rate of a pre-existing cancer or tumor or esophageal epithelial proliferation disease.

As used herein, the terms “improved therapeutic outcome” and “enhanced therapeutic efficacy”, relative to cancer or an esophageal epithelial proliferation disease refers to a slowing or diminution of the growth of cancer cells or a solid tumor or esophageal disease, or a reduction in the total number of cancer cells or total tumor burden. An “improved therapeutic outcome” or “enhanced therapeutic efficacy” means there is an improvement in the condition of the individual according to any clinically acceptable criteria, including reversal of an established tumor, an increase in life expectancy or an improvement in quality of life.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5 (carboxyhydroxyl¬methyl) uracil, 5-fluorouracil, 5 bromouracil, 5-carboxymethylaminomethyl 2 thiouracil, 5 carboxymethyl¬aminomethyluracil, dihydrouracil, inosine, N6 isopentenyladenine, 1 methyladenine, 1-methylpseudo¬uracil, 1 methylguanine, 1 methylinosine, 2,2-dimethyl¬guanine, 2 methyladenine, 2 methylguanine, 3-methyl¬cytosine, 5 methylcytosine, N6 methyladenine, 7 methylguanine, 5 methylaminomethyluracil, 5-methoxy-amino¬methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

The term “wild-type” refers to a gene or gene product (e.g., SFRP1) isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product. A “biologically active fragment or variant” refers to a modified or mutant gene or gene product that exhibits biological activity of the wild-type gene or gene product (e.g., an activity that is 20-200% the activity of the wild type gene or gene product, however, the disclosure is not limited to this amount of activity, the activity may be more than 200% or less than 20% of the wild type gene or gene product).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein (e.g., SFTP1) is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, recombinant polypeptides can be expressed in bacterial host cells and the polypeptides purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

1 “Sequence identity,” “% sequence identity” and the like with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “vector” or “expression vector” is a replicon, such as plasmid, phage, virus, or cosmid, to which another DNA segment, e.g., an “insert,” may be attached or incorporated so as to bring about the replication of the attached segment in a cell.

Compositions may be formulated for any appropriate manner of administration, and thus administered including, but not limited to, intravenously, arterially, intradermally, intramuscularly, intraperitoneally, subcutaneously, ocularly, sublingually, orally, intranasally, intervaginally, intracranially, intraperitoneally, epicutaneously, intraspinally, intracerebrally, and transdermally. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage.

The terms “effective amount” or “therapeutically effective amount,” as used herein, refer to a sufficient amount of a composition described herein being administered which will relieve to some extent one or more of the symptoms of the disease or condition being treated (e.g., cancer and/or tumor proliferation and/or growth and/or esophageal epithelial proliferation disease). The result can be reduction and/or alleviation of the signs, symptoms, or causes of a disease, or any other desired alteration of a biological system. For example, an “effective amount” for therapeutic uses is the amount of a compositions required to attenuate and/or modulate cancer and/or tumor proliferation and/or growth and/or esophageal epithelial proliferation disease.

The amount of the composition required for use in treatment or prevention will vary not only with the particular composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, in some embodiments, useful dosages of compositions required to attenuate and/or modulate cancer and/or tumor proliferation and/or growth and/or esophageal epithelial proliferation disease can be determined by comparing their in vitro activity, and in vivo activity in animal models.

Dosage amount and interval may be adjusted individually to provide plasma levels of a therapeutic composition (e.g., a SFRP1 activator or a composition thereof or SFRP1, a biologically active fragment or variant thereof, or a nucleic acid encoding SFRP1 or a biologically active fragment or variant thereof) which are sufficient to maintain the modulating effects, or minimal effective concentration (MEC). The MEC will vary for each composition disclosed herein but can be estimated from in vivo and/or in vitro data. Dosages necessary to achieve the MEC will depend on individual characteristics and route of administration. However, bioassays can be used to determine plasma concentrations. Dosage intervals can also be determined using MEC value. Compositions may be administered using a regimen, which maintains plasma levels above the MEC for 10-90% of the time, preferably between 30-90% and most preferably between 50-90%. In cases of local administration or selective uptake, the effective local concentration of the composition may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may also be used in veterinary medicine for non-human subjects.

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition. For example, SFRP1 of the disclosure may be generated using any expression system known with the resulting C3d being substantially purified therefrom.

The present disclosure provides for DNA segments encoding the proteins (e.g., SFRP1 or biologically active fragment thereof and/or STAT3 or inhibitor thereof) and nucleic acids (e.g., nucleic acids and vectors encoding the disclosed proteins, gene silencing oligonucleotides, etc.) disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The nucleic acid encoding the proteins (e.g., SFRP1 or biologically active fragment thereof and/or STAT3 or inhibitor thereof, etc.) disclosed herein may be any nucleic acid including DNA, RNA, or combinations thereof. In some embodiments, the nucleic acid encoding the proteins (e.g., SFRP1 or biologically active fragment thereof and/or STAT3 or inhibitor thereof, etc.) comprises a messenger RNA or a vector.

In certain embodiments, engineering the nucleic acid for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

The present disclosure further provides engineered, non-naturally occurring vectors and vector systems, which can encode the proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides, etc.) disclosed herein. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding the proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides, etc.) disclosed herein into cells, tissues, or a subject. Such methods can be used to administer nucleic acids encoding the proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides) disclosed herein to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that the nucleic acid encoding the proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides) disclosed herein may be removed from the cells under certain conditions.

A variety of viral constructs may be used to deliver the proteins and nucleic acids (e.g., nucleic acids and vectors encoding the disclosed proteins, gene silencing oligonucleotides) disclosed herein to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides) disclosed herein is contained in a plasmid vector that allows expression of the protein(s) and subsequent isolation and purification of the protein produced by the recombinant vector. Accordingly, the proteins can be purified following expression, obtained by chemical synthesis, or obtained by recombinant methods.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ube (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of an RNA or protein can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various ubiquitous as well as tissue-specific promoters and tumor-specific are commercially available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein or RNA operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., esophageal epithelial cells or subpopulation thereof) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The present proteins (e.g., SFRP1 or biologically active fragment thereof and/or STAT3 or inhibitor thereof, etc.), nucleic acids encoding these proteins, and compositions comprising the proteins and/or nucleic acids described herein may be delivered by any suitable means. In certain embodiments, they are delivered in vivo, as described above. In other embodiments, they are delivered to isolated/cultured cells in vitro (e.g., to provide modified cells useful for in vivo delivery to patients afflicted with a disease or condition).

As described above the proteins (e.g., SFRP1 or biologically active fragment thereof and/or STAT3 or inhibitor thereof, etc.) or nucleic acids encoding thereof may be introduced into cells by methods known in the art. In some embodiments, the cell is a mammalian cell. In some embodiments, the cell is a mammalian cell (e.g., a cell of a non-human primate or a human cell). Accordingly, provided herein are cells comprising the disclosed proteins (e.g., SFRP1 or biologically active fragment thereof and/or STAT3 or inhibitor thereof) or nucleic acids encoding thereof.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes proteins (e.g., SFRP1 or biologically active fragment thereof and/or STAT3 or inhibitor thereof, etc.) and nucleic acids (e.g., nucleic acids and vectors encoding the disclosed proteins, gene silencing oligonucleotides) disclosed herein is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the vector can be delivered by any method appropriate for introducing nucleic acids into a cell.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

In some embodiments, a composition comprising SFRP1 or biologically active fragment thereof and/or STAT3 or inhibitor thereof comprises between 0.1 and 500 μg of SFRP1 or biologically active fragment thereof and/or STAT3 or inhibitor thereof. However, the present disclosure is not limited to these amounts. For example, in some embodiments, more than 500 g of SFRP1 or biologically active fragment thereof and/or STAT3 or inhibitor thereof is present in a composition for administration to a subject. In some embodiments, less than 0.1 g of SFRP1 or biologically active fragment thereof and/or STAT3 or inhibitor thereof is present in a composition for administration to a subject.

In some embodiments, each dose comprises an amount of SFRP1 or biologically active fragment thereof and/or STAT3 or inhibitor thereof sufficient to generate the desired cell mediated immune response. An effective amount of SFRP1 or biologically active fragment thereof and/or STAT3 or inhibitor thereof in a dose need not be quantified, as long as the amount SFRP1 or biologically active fragment thereof and/or STAT3 or inhibitor thereof generates the desired response in a subject when administered to the subject. An optimal amount for a particular administration (e.g., to induce a desired response) can be ascertained by one of skill in the art using standard assays and procedures (e.g., those described and/or referenced herein). In some embodiments, it is expected that each dose (e.g., of a composition comprising a SFRP1 or biologically active fragment thereof and/or STAT3 or inhibitor thereof) comprises 0.05-5000 μg of SFRP1 or biologically active fragment thereof and/or STAT3 or inhibitor thereof, in some embodiments, each dose will comprise 1-500 μg, in some embodiments, each dose will comprise 350-750 μg, in some embodiments, each dose will comprise 50-200 μg, in some embodiments, each dose will comprise 25-75 μg. The specific dose level and frequency of dosage for any particular patient may be varied and will depend upon a variety of factors including the activity of the specific composition employed, the metabolic stability and length of action of the compound, the age, body weight, general health, sex, diet, mode and time of administration, rate of excretion, drug combination, the severity of the particular condition, and the host undergoing therapy.

Composition described herein can be evaluated for efficacy and toxicity using known methods. For example, the toxicology of a particular composition can be established by determining in vitro toxicity towards a cell line, such as a mammalian, and preferably human, cell line. The results of such studies are often predictive of toxicity in animals, such as mammals, or more specifically, humans. Alternatively, the toxicity of particular composition in an animal model, such as mice, rats, rabbits, dogs, or monkeys, may be determined using known methods. As detailed herein, C3d compositions displayed little to no toxicity indicating relatively high dosing may be possible. The efficacy of a particular composition may be established using several recognized methods, such as in vitro methods, animal models, or human clinical trials. When selecting a model to determine efficacy, the skilled artisan can be guided by the state of the art to choose an appropriate model, dose, route of administration and/or regime. The efficacy of C3d compositions is shown in the examples.

An effective amount of a composition of the disclosure may be administered alone or in combination with at least one additional therapeutic agent. In some embodiments, the at least one additional therapeutic agent is administered prior to, concomitantly with, or following administration of the composition (e.g., SFRP1 or biologically active fragment thereof and/or STAT3 or inhibitor thereof). The additional therapeutic agent may include, for example, a corticosteroid, an immunosuppressant, rituximab, anti-inflammatory agent, an antibiotic, an opioid antagonist, a vitamin or nutritional supplement, or any combination thereof. The choice of additional therapeutic agents will not only depend on the particular disease or disorder but also the nature and/or symptoms of the disease and the age and condition of the patient.

The disease or disorder may be characterized by abnormal cell growth and/or abnormal esophageal epithelial cell growth and/or tumor proliferation and/or growth. As such, the disease or disorder may comprise esophageal epithelial proliferation disease or cancer or other type malignancy described herein.

A composition comprising SFRP1 or biologically active fragment thereof and/or STAT3 or inhibitor thereof disclosed herein may be used in combination with, preceding, following, or in lieu of other treatments and/or therapies. For example, a subject to whom a composition comprising SFRP1 or biologically active fragment thereof and/or STAT3 or inhibitor thereof of the disclosure is administered may be treated (e.g., previously, concurrently, or subsequently) with surgical intervention, chemotherapy, radiation therapies and/or other forms of immunotherapy that are commonly used and well known in the art to treat aberrant cell proliferation disease.

The following examples further illustrate the disclosure but should not be construed as in any way limiting its scope.

### EXAMPLES

### Example 1—Identification of Genes Involved in IL-13 Induced Esophageal Proliferation

### Materials and Methods

RNAseq pre-processing and quality control of Human subjects—NL (healthy control patients) were defined as having no history of EoE diagnosis, 0 esophageal eosinophils per high-power field (HPF) and no evidence of esophagitis within distal esophageal biopsies obtained during the same endoscopy procedure as the analyzed samples. EoE was defined as described in the recent consensus guidelines 39, 40. Specifically, patients needed to have ≥15 cosinophils in at least 1 high-power field (Eos/hpf) in a distal esophageal biopsy with other causes of esophageal eosinophilia excluded and having no response to acid suppression. RNAseq files were downloaded from (GSE and the FASTQC program and Trimmomatic tools were used to examine the quality of raw reads and filtering poor quality reads, respectively. Genome indexing was performed using Bowtie2 and the reads were aligned to the human reference genome (GRCh38) using HiSAT2 program with the default options. Read counts were generated using the feature-counts function from the subRead package. Downstream analysis was performed using DEseq2 and EdgeR in R (R Core Team, Vienna, Austria). DEGs were filtered using p-value ≤0.05, and absolute log 2-fold-change (abs Log 2FC)>1.

EPC2-ALI culture—hTERT immortalized human esophageal epithelial keratinocytes (hTERT-EPC2) were cultured under air-liquid-interface conditions as described 9, 41. In brief, EPC2-hTERT cells were seeded onto permeable (0.4 μm) transwell support (Corning Incorporated, Corning, NY, USA) and grown to confluence while fully submerged in low-calcium ([Ca2+]=0.09 mM) keratinocyte serum-free media (K-SFM) (Life Technologies; Carlsbad, CA). Epithelial differentiation was induced by culturing submerged cell monolayers in high-calcium K-SFM ([Ca2+]=1.8 mM) for 4 days (day 3 to 7). Cells were then exposed for 5 days (day 7 to 12) at ALI by removing cell media from the top chamber, to induce stratification.

Primary esophageal cell cultured from biopsies—Primary esophageal cell cultures were generated as described by Ferrer-Torres et al, 2022. Primary esophageal cells cultured from biopsies. Esophageal biopsies were collected in HYENAC media from males and females who underwent endoscopy. Biopsies were finely minced and seeded onto irradiated NIH 3T3-J2 mouse embryonic fibroblast (MEF) cells (7.5×104 cells per well; Kerafast; Catalog: EF3003) in a 6 well plate and were maintained and split upon reaching confluency for 8 passages. For western blot and RT-qPCR studies, primary esophageal cells were stimulated with IL-13 (100 ng/ml) for 0-120 minutes and 48 hours and were collected and lysed for protein (NP40 lysis buffer with protease and phosphatase inhibitor) and mRNA (Tripure with Phenol chloroform) as described below. For immunofluorescence studies, primary esophageal cells were grown on MEFs which were cultured on top of 12 mm siliconized glass coverslides in 12-well tissue culture plates. Esophageal cells were seeded at high-density (500,000 cells) and were cultured for 14 days before fixation, primary and secondary antibody staining, and fluorescent protein detection using confocal microscopy as described below. Cells were immediately processed for culture or cryopreserved.

RNAseq pre-processing and quality control of esophageal epithelial keratinocytes—For RNAseq, EPC2-ALI were cultured and stimulated with Vehicle or IL-13 (100 ng/mL) for 4 hours or 48 hours. RNA was isolated from cells with the RNeasy Kit (Qiagen, Germantown, Md), according to the manufacturer's protocol. RNA quality was assessed by using the Agilent 2100 Expert Bioanalyzer (Agilent Technologies, Santa Clara, Calif), and only the samples with RNA integrity numbers of greater than 8 were processed for sequencing. RNA samples were subjected to RNA-seq at the Cincinnati Children's Hospital Medical Center (CCHMC) Gene Discovery and Genetic Variation Core, as described 42. The same pre-processing steps were performed as described in RNAseq analysis of Human subjects until read counts were generated using subRead. The IDEP 9.1 webtool and rStudio DeSeq2 was used to analyze read counts and identify the differentially expressed genes (DEGs). DEGs were filtered using abs Log 2FC)>1.

RNAseq analysis of esophageal biopsies and EPC2-ALI—GO biological processes were identified using DAVID Bioinformatics Resources 6.8. Common and unique DEGs were identified via DeSeq2 and represented as Venn diagrams (https://bioinformatics.psb.ugent.be/webtools/Venn/). Putative transcription factor targets analysis was performed using the ChIP-X Enrichment Analysis (ChEA), which is a gene-set enrichment analysis tool to probe whether DEG lists contain putative targets of transcription factors. ChEA datasets for STAT1, STAT3, STAT4, STAT5a, and STAT6 were used and mapped onto the 82 common DEGs identified through the RNAseq analysis. 82 DEGs were also mapped onto the “Cell Proliferation” gene ontology term (GO) to identify DEGs which were putative STAT3 targets and associated with “Cell Proliferation.”

Single-cell RNAseq analysis of esophageal epithelium in homeostasis and disease—Data (UMI) count matrices, feature counts, and barcodes for each sample were acquired from NCBI GEO (GSE201153) and uploaded to Seurat v4 for downstream analysis. In brief, for each sample, the standard Seurat “Introduction to scRNA-seq Integration” vignette was used to perform downstream analysis. The data was processed through the standard pre-processing workflow including quality control (QC) metrics, such as removal of low-quality cells, empty droplets, cell doublets and cell multiplets. Cells with unique feature counts greater than 2500 or less than 200, and cells with >5% mitochondrial counts were filtered out. The UMI count matrix was then normalized using Log Normalize and scaled using a scale factor of 10000, according to the standard Seurat pipeline. Samples were then integrated by all 10 samples using the IntegrateData function, to compare the cellular populations in an unbiased manner. Initial clustering was performed using the FindClusters function with a resolution of 0.5, to identify the top 20 principal components and detect predominant cell types, including epithelial cells, mast cells, fibroblasts, lymphocytes, myeloid cells, and endothelial cells. FindConservedMarkers was employed to identify the canonical cell type-specific markers that are conserved across samples. Epithelial cells were isolated for the secondary clustering to identify 12 epithelial subpopulations (resolution: 0.5), broadly categorized into quiescent, proliferating, transdifferentiated 1, transdifferentiated 2, differentiated HI, and differentiated LO clusters; cell type-specific marker gene identification was performed the same way as the initial clustering. A seventh epithelial subpopulation was mapped onto “Basal” and “Suprabasal” epithelial markers to determine the phenotype of this cluster. Further, differential gene expression analysis was performed using DeSeq2 to identify differentially expressed genes in the new epithelial subcluster. StringDB and Cytoscape were used to represent the gene networks present in the novel epithelial subcluster.

Data visualization—DEG comparisons were visualized using the Set Comparison Appyter from the Ma'ayan Lab (https://appyters.maayanlab.cloud/#/CompareSets). Network analysis was performed in Cytoscape3.7.1. Figures were constructed using Inkscape 1.1.1 and Adobe Illustrator 26.0.2.

Lentiviral transduction of EPC2 cells—EPC2 cells were transduced at 60-70% confluency with lentiviral particles containing Mission® STAT3 shRNA (TRCN0000329887, Sigma; St. Louis, MO, USA) or Mission® non-target control shRNA (Sigma; St. Louis, MO, USA). Lentiviral particles were incubated with EPC2 cells for 6 hours. All the viral particles were added in the presence of 5 μg/mL Hexadimethrine Bromide (Polybrene®) (Sigma; St. Louis, MO, USA). During the first hour of incubation, cells were spun down at 1000*g for 1 hour at room temperature. 6 hours following transduction cells were put in fresh KSFM media, and 24 hours later media containing 1 μg/mL of Puromycin (Thermo Fisher Scientific Incorporated; Rockford, IL, USA) was used for selection. Cells were grown under selective pressure and cultured as regular EPC2 cells. Stable knockdown for STAT3 was demonstrated by western blot and RNAseq analyses.

Krt5-rtTA×tetO-IL-13 murine model of EoE-like epithelial remodeling—Krt5-rtTA mice were backcrossed on the tetO-IL-13 background. Mice where rtTA is specifically expressed in esophageal Krt5+ cells under the tissue specific promoter are crossed with mice with a tetracycline response element with IL-13 downstream. The crossed mice would allowed tissue-specific spatial and temporal regulation of IL-13 gene expression. 8-10-week-old mice received doxycycline chow (625 ppm, Envigo) daily for two weeks or four weeks to activate esophageal IL-13 expression.

Quantitative PCR—RNA was extracted from EPC2-ALI cells by using the Quick-RNA Microprep Kit (Zymo Research Corporation) according to the manufacturer's protocol. Purified RNA (500 ng) was DNase treated and reverse transcribed to cDNA by using Superscript II RNase H Reverse Transcriptase (Thermo Fisher Scientific, Rockford, Ill), according to the manufacturer's instructions. cDNA for ANO1, CAPN14, CCL26, TP63, SFRP1, STAT3 and hypoxanthine phosphoribosyltransferase (HPRT) was quantified by using real-time PCR with the IQ SYBR Green Supermix (Bio-Rad Laboratories, Hercules, Calif) with the CFX96 Touch Real-Time PCR Detection System (Bio-Rad Laboratories). Quantitative PCR (qPCR) analyses were performed by using Bio-Rad CFX Manager Software (version 3.1; Bio-Rad Laboratories), and the results were normalized with HPRT amplified from the same cDNA mix and expressed as fold induction (2\hat{ }-(ΔΔCT). Primers used for amplification were:

| Primers | | Sequence (5′ to 3′) |
|---|---|---|
| SFRP1 | Forward | CTCAACAAGAACTGCCACGC |
| | | (SEQ ID NO.: 1) |
| | Reverse | CTCGTTGTCACAGGGAGGA |
| | | (SEQ ID NO.: 2) |
| ANO1 | Forward | GAGCCAAAGACATCGGAATC |
| | | TG (SEQ ID NO.: 3) |
| | Reverse | TGAAGGAGATCACGAAGGCA |
| | | T (SEQ ID NO.: 4) |
| TP63 | Forward | AAGCGCCCGTTTCGTCAG |
| | | (SEQ ID NO.: 5) |
| | Reverse | TGTGCTGAGGAAGGTACTGC |
| | | (SEQ ID NO.: 6) |
| CCL26 | Forward | AGTGCTGCTTCTGTTCCCAA |
| | | (SEQ ID NO.: 7) |
| | Reverse | AAGGTGGAGACTCAGGAGGG |
| | | (SEQ ID NO.: 8) |
| CAPN14 | Forward | CAATGACGGAGAATTCTGGA |
| | | TG (SEQ ID NO.: 9) |
| | Reverse | GTTCTTCCAAAATGTGTCCT |
| | | GC (SEQ ID NO.: 10) |
| HPRT | Forward | CAGACTGAAGAGCTATTGTA |
| | | ATG (SEQ ID NO.: 11) |
| | Reverse | CCAGTGTCAATTATATCTTC |
| | | CAC (SEQ ID NO.: 12) |

Western blot—EPC2 ALI cells and primary esophageal cells were lysed following stimulation, using a protein extraction reagent (10% Glycerol, 20 mM Tris HCl pH7, 137 mM NaCl, 2 mM EDTA, 1% NP-40 in H2O) with Halt™ protease inhibitor cocktail (Thermo Fisher Scientific Incorporated; Rockford, IL, USA) and PhosSTOP phosphatase inhibitor cocktail tablet (Roche Diagnostics; Mannheim, Germany). 25 g of protein extract were separated on a 4%-12% Bis-Tris gel and transferred to a nitrocellulose membrane (Life Technologies; Carlsbad, CA). The following primary antibodies were used: anti-Phospho-STAT3 (Tyr705) (1:500, #9145, Cell Signaling Technology; Danvers, MA), anti-Phospho-STAT3 (Ser727) (1:500, #, Cell Signaling Technology; Danvers, MA), anti-STAT3 (1:1000, #4094, Cell Signaling Technology; Danvers, MA), anti-Phospho-STAT6 (Y641) (1:1000, #9361, Cell Signaling Technology; Danvers, MA), anti-STAT6 (1:1000, #9362, Cell Signaling Technology; Danvers, MA), anti-SFRP1 (1:500, #3534, Cell Siganling Technology; Danvers, MA) and anti-β-Actin (1:5000, #4970, Cell Signaling Technology; Danvers, MA). Anti-rabbit IgG HRP-linked antibody (1:10,000, #7074, Cell Signaling Technology; Danvers, MA) was used as the secondary antibody. For chemiluminescent detection of proteins, SuperSignal™ West Femto Maximum Sensitivity Substrate was diluted 1:5 in SuperSignal™ West Pico PLUS Chemiluminscence Substrate detection reagent (34094 and 34579, Thermo Scientific™, Life Technologies I Carlsbad, CA, USA).

Histopathological Analysis—EPC2 cells were cultured in ALI conditions for 5 days and treated for 48 hours with their respective stimulation conditions. EPC2-ALI cells were then fixed on Transwell support with 4% paraformaldehyde (PFA) for 1 hour at room-temperature and underwent a series of ethanol (EtOH) washes (30% EtOH 4 50% EtOH 4 70% EtOH) for seven minutes per wash. Esophageal tissues from mice were fixed in 4% PFA overnight at 4° C. and were then submerged in 70% EtOH. Formalin-fixed Transwell membranes with EPC2 cells and esophageal tissues were embedded in paraffin and sectioned at 5 μm using a microtome. Sections were mounted on slides, deparaffinized and rehydrated using standard histological techniques. Hematoxylin & Eosin (H&E) staining was used to study esophageal eosinophilia and esophageal remodeling (DIS, BZH, epithelial thickness). Stained slides were imaged with the Olympus DP-72 microscope and CellSens standard software (Olympus, Semrock, New York, NY). FIJI ImageJ was used for histologic and morphometric analysis. DIS was measured in nanometers, and ten intercellular space measurements were averaged to obtain a representative measurement per mouse esophagus. Total epithelial thickness was measured in micrometers (μM) and ten different epithelial thickness measurements were taken from various regions of the mouse esophagus to obtain a representative measurement of total epithelial thickness. The same method was used to measure BZH; the average of ten measurements of basal zone (basal layers of epithelium) were taken and then divided by total epithelial thickness to obtain the percent basal zone of epithelium.

Immunofluorescence Staining—Formalin-fixed and paraffin-embedded (FFPE) esophageal tissues on glass slides were deparaffinized and rehydrated using standard histological techniques. Slides were then permeabilized in Tris-EDTA (1 mM, pH 9.0) with 0.1% Tween-20, and antigen exposure performed at 125° C. for 30 seconds in a decloaking chamber. Slides were washed in 1×PBS twice for 5 minutes each wash and then incubated blocked in 4% Normal donkey serum (Jackson ImmunoResearch; Catalog: 017-000-121), diluted in 1×PBS, for 1 hour to reduce non-specific hydrophobic interaction between primary antibody and the tissue. This was followed by overnight incubation of primary antibodies diluted in 4% normal donkey serum: anti-Krt15 (Cytokeratin 15 monoclonal antibody (LHK15); Thermo Fisher Scientific; Catalog: MA5-11344), anti-KRT4 (Cytokeratin 4 polyclonal antibody; Thermo Fisher Scientific; Catalog: 16572-1-AP), anti-K167 (Ki-67 monoclonal antibody, Thermo Fisher Scientific; Catalog: 14-5698-82). Slides were then washed and incubated with secondary antibody at RT for 1 hour. Slides were mounted with DAPI Fluoromount-G (SouthernBiotech, Birmingham, Ala) mounting solution. Fluorescence imaging was performed with the Zeiss Apotome fluorescent microscope (Carl Zeiss Meditec, Dublin, California) with Nikon Elements software and ImageJ software (National Institutes of Health, Bethesda, Md).

Immunohistochemistry Staining—Formalin-fixed and paraffin-embedded (FFPE) esophageal tissues on glass slides were deparaffinized and rehydrated using standard histological techniques. Slides were then permeabilized in Tris-EDTA (1 mM, pH 9.0) with 0.1% Tween-20, and antigen exposure performed at 125° C. for 30 seconds in a decloaking chamber. Slides were washed in 1×PBS twice for 5 minutes each wash and then incubated in 3% hydrogen peroxide (H2O2) for 30 minutes. Sides were washed again in 1×PBS twice for 5 minutes and blocked in 4% Normal Donkey Serum for 1 hour to reduce non-specific hydrophobic interaction between primary antibody and the tissue. To block endogenous biotin binding, samples were incubated in 1-3 drops of avidin blocking reagent for 15 minutes. To block subsequent binding to avidin, samples were incubated with 1-3 drops of biotin blocking reagent for 15 minutes, followed by overnight incubation of primary antibody anti-STAT3 (1:1000, #30835, Cell Signaling Technology, Danvers, MA) at 4° C. The next day, samples were washed three times with 1×PBS for 5 minutes each and incubated with biotinylated anti-rabbit IgG secondary antibody for 60 minutes. Slides were rinsed in 1×PBS 3 times and incubated with ABC reagents (Vectastain PK-6100, Vector Labs, Burlingame, California, USA) for 30 minutes at room temperature. DAB peroxidase substrate was prepared immediately before use (Vectastain, SK-4100, Vector Labs, Burlingame, California, USA), applied to the slides and observed for brown staining. Slides were added to tap water to stop the reaction and counterstained with Harris Modified Hematoxylin Solution for 4 minutes (#HHS32, Sigma Aldrich, Saint Louis, MO, USA). Slides were then dehydrated using standard histological techniques and mounted with Cytoseal mounting media (C860G35, ThermoFisher Scientific, Kalamazoo, MI, USA).

Masson's Trichrome Histopathological Staining—PFA-fixed and paraffin-embedded (FFPE) murine esophageal tissue sections were deparaffinized according to standard histological procedures. The Masson's Trichrome staining was performed according to manufacturer's protocol and instructions (Trichrome Stain Kit, Connective Tissue Stain, ab150686; Abeam).

Quantification of 5-bromo-2′-deoxyuridine-positive EPC2-ALI cells—5-Bromo-2′-deoxyuridine (BrdU) was obtained from Sigma-Aldrich. EPC2-ALICTRL and EPC2-ALIASTAT3 cells were cultured as described above, treated for 48 hours with Vehicle or IL-13 (100 ng/mL), and then exposed to BrdU (10 mol/L) in dimethyl sulfoxide for 2 hours at 37° C. Cells were then fixed on support for 2 hours with 4% PFA at RT. Fixed membranes were then processed, embedded, and sectioned as described above. Immunofluorescence staining was performed using methods described in the previous section. Samples were stained for detection of BrdU+ cells by using 2.5 μg/L G3G4 anti-BrdU antibody (Developmental Studies Hybridoma Bank, Iowa City, Iowa) and 4′-6-diamidino-2-phenylindole dihydrochloride (DAPI). The number of BrdU+-labeled cells were quantitated under a 20×objective by using the Zeiss Apotome fluorescent microscope (Carl Zeiss Meditec). The BrdU+-labeled cells were quantified as the number of BrdU+-labeled cells per total linear length of filter-attached EPC2-ALI cells using FIJI ImageJ and calculated based on the ratio of BrdU+ cells per millimeter of membrane. Three filters per condition from 3 individual experiments were quantitated.

Quantification of Ki67-positive EPC2-ALI cells—EPC2-ALI cells were cultured in the presence or absence of their respective stimulations for 48 hours. For immunofluorescence (IF) staining, formalin- or paraformaldehyde-fixed, paraffin-embedded transwell sections were sectioned, mounted on slides, and deparaffinized by using standard histologic procedures. Slides were then permeabilized in Tris-EDTA (1 mmol/L, pH 9.0) with 0.1% Tween-20, and antigen exposure was performed at 125° C. for 30 seconds in a decloaking chamber by using a pressure cooker. Slides were then blocked by 10% normal donkey serum for 1 hour, followed by overnight incubation of primary antibodies diluted in 10% normal donkey serum at a final concentration of 1 μg/mL: anti-Ki67 (1:1000; clone: SolA15; Invitrogen), slides were then washed and incubated with secondary antibody at RT for 1 hour. Slides were mounted with 4′-6-diamidino-2-phenylindole dihydrochloride Fluoromount-G (SouthernBiotech, Birmingham, Ala) mounting solution. Fluorescent imaging was performed with the Zeiss Apotome fluorescent microscope (Carl Zeiss Meditec, Dublin, Calif) using Nikon Elements software. Ki67+ cells were quantified as the number of Ki67+-labeled cells per total linear length of filter-attached EPC2-ALI cells using FIJI ImageJ. Three filters per condition from 3 individual experiments were quantitated.

Statistical Analysis—Statistical significance of EPC2-ALI samples were established using unpaired t-test (two-tailed), or two-way ANOVA when there was more than one variable. For non-normally distributed data from patient biopsies, Mann-Whitney test was used. Graphs and statistical analyses were performed using GraphPad Prism 9.1 (GraphPad Software Incorporated, La Jolla, CA, USA).

### Enrichment of STAT3-Regulated Genes in EoE and IL-13-Stimulated EPC2 ALI.

To identify the key genes involved in IL-13-induced esophageal proliferation, the DEGs identified from RNAseq analysis of IL-13-stimulated EPC2-ALI cells were mapped onto the DEGs biopsy samples from normal healthy controls and pediatric EoE patients (GSE58640; n=6 controls and n=9 EoE). Analyses of differential gene expression in biopsy samples from normal healthy controls and pediatric EoE patients identified 1470 dysregulated genes (absolute Log FC>1; FDR: adjusted p-value <0.05). GO gene ontology analyses identified significant enrichment of genes associated with immune and inflammatory regulation (CCL26, CDH26), epithelial cell proliferation (ANO1, SLC26A4-AS1), and epithelial barrier regulation (SLC9A3, ALOX15, POSTN)26. RNAseq analysis of EPC2-ALI stimulated with Vehicle or IL-13 for 4 hours and 48 hours identified 1563 DEGs (absolute Log FC>1) and 1425 DEGs respectively (absolute Log FC>1; See FIG. 1A). Notably, when the 1470 DEGs identified in pediatric EoE were mapped onto 1425 IL-13-induced DEGs in epithelial cells 82 common DEGs were identified referred to herein as esophageal epithelial-specific EoE genes (See FIG. 1B). The 82 esophageal epithelial-specific EoE DEGs revealed significant enrichment of pathways involving “cytokine-cytokine receptor interaction” and “Interleukin-4 and Interleukin-13 signaling”. Key genes associated with “cytokine-cytokine receptor interaction” and “Interleukin-4 and Interleukin-13 signaling” pathways included C—C chemokine CCL24 (eotaxin 2), CXCL14, and SOCS1 (suppressor of cytokine signaling-1). Other highly enriched pathways in the 82 DEGs included “Wnt signaling pathway” (fold enrichment: 4.30), “Signaling by WNT” (fold enrichment: 2.05), and “Pathways in cancer” (fold enrichment: 1.35). To gain insight into the transcriptional regulation of the 82 esophageal epithelial-specific EoE DEGs ChEA TF gene sets were utilized and revealed that the 82 common DEGs were enriched for putative STAT protein targets including STAT1, STAT3, STAT4, STAT5a and STAT6 (FIG. 1C). Of the 82 common DEGs, n=32 (39%) were putative STAT3 targets (FIG. 1D). Collectively, this disclosure provides that esophageal epithelial-specific EoE DEGs are enriched for pathways associated with allergic inflammation and Wnt signaling pathways and are enriched for genes that are predominantly putative STAT3 targets.

IL-13 Activates STAT3 in EPC2-ALI and Primary Esophageal Cells Derived from Patient Biopsies.

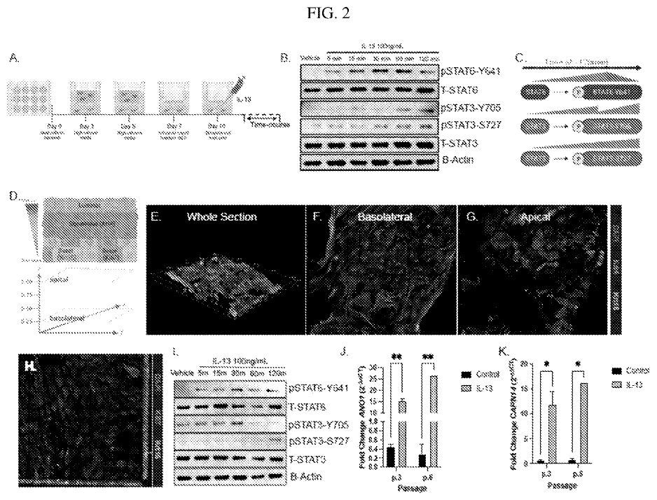

To gain insight into the involvement of STAT3 in IL-13-induced esophageal epithelial signaling and proliferation, two in vitro culture systems were utilized, the EPC2-ALI and primary esophageal cell culture derived from patient biopsies (9, 41, 43). Western blot analyses of IL-13-stimulated EPC2-ALI cells revealed a significant time-dependent phosphorylation and activation of STAT6 with pSTAT6 observed at 5-minute post simulation and peaking by 60 minutes (FIG. 2A-2C). IL-13 also induced a significant time-dependent increase in phosphorylation of STAT3 at tyrosine 705 (Y705) and serine 727 (S727). (FIG. 2A-2C). Notably, the kinetics of STAT3 phosphorylation differed from that of STAT6, in that STAT3 underwent a gradual and constitutive phosphorylation reaching maximal at 60- and 120-minutes post stimulation (FIG. 2B). Next, a complex 3D primary esophageal cell culture of human esophagus derived from esophageal biopsy43 was generated. Immunofluorescence analyses showed that the complex 3D esophageal cell culture consists of immature basal cells (FIG. 2D-2F; Krt15+) at the base of the culture and a suprabasal (FIG. 2D-2E; Krt4+) zone consisting of maturing cells apically, representing increasing maturation of esophageal cells from the basolateral to apical surface. Notably, colocalization of Krt15 and Ki67, indicated the presence of proliferating basal cells (FIG. 2H; Krt15+, Ki67+). IL-13 stimulation of patient-derived primary esophageal cells led to phosphorylation and activation of STAT3 and STAT6 (FIG. 2I). IL-13-induction of STAT6 (Y641) peaked at 30 minutes and plateaued by 60 minutes. IL-13-induced rapid phosphorylation of STAT3 at Y705 residue reaching maximal by 30 minutes and declining over the 120-minute time course. IL-13 phosphorylation of S727 residue was delayed and gradually increased over the 120-minute time course (FIG. 2I). IL-13 stimulation of primary esophageal cells at different passages induced increased mRNA expression of canonical EoE markers, ANO1 and CCL26(FIG. 2J-2K) (9, 26). Thus, the disclosure provides that IL-13-induces phosphorylation and activation of STAT6 (Y641) and STAT3 (Y705 and S727) in esophageal epithelial cells.

Mice with Inducible Overexpression of IL-13 in Esophageal Epithelial Cells Exhibit Epithelial Remodeling and Increased STAT3 Expression.

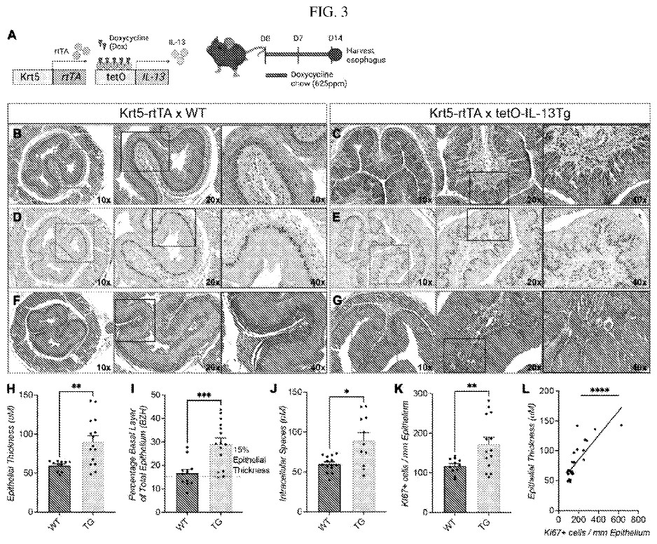

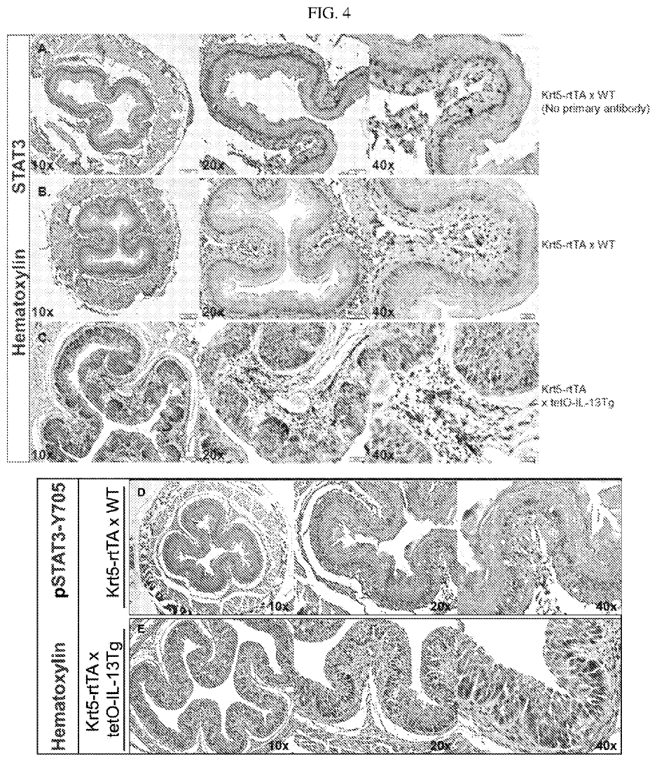

To validate the in vitro approaches and to identify the role of STAT3 in esophageal epithelial remodeling, a murine model using Krt5-rtTA tetO-IL-13Tg mice was used where IL-13 is induced in esophageal epithelial cells upon receiving doxycycline chow ([Dox+], 625 ppm) for two weeks 44. (FIG. 3A). Dox treatment of Krt5-rtTA×tetO-IL-13Tg mice, and not Krt5-rtTA×WT counterparts, induced increased esophageal mRNA expression of IL-13, and was associated with esophageal epithelial remodeling including increased total esophageal epithelial thickness, percentage basal zone to total epithelial thickness and DIS (FIG. 3B-3F). Krt5-rtTA×tetO-IL-13Tg mice which did not receive 2-weeks of Dox (Krt5-rtTA×tetO-IL-13Tg, Dox−) resembled the histological and morphometric characteristics like that observed in Dox-treated Krt5-rtTA×WT mice. Immunohistochemistry analysis probing for Ki-67 revealed that Dox+ Krt5-rtTA×tetO-IL-13Tg mice had increased esophageal epithelial proliferation (Ki-67+ cells/mm epithelium) compared to their WT counterparts (FIG. 3G-3I), and Ki-67+ cells/mm epithelium positively correlated with total epithelial thickness (r=0.7926, p<0.0001; FIG. 3J). Fibrosis staining revealed a dense band of outer-epithelial collagen in WT mice in contrast to inter-epithelial collagen deposition in Dox+ Krt5-rtTA×tetO-IL-13Tg (FIG. 3K-3L). Notably, increased esophageal epithelial proliferation was associated with increased total STAT3 protein expression in Dox+ Krt5-rtTA×tetO-IL-13Tg compared to WT counterparts or no-primary antibody negative controls (FIG. 4A-4E). Accordingly, the disclosure provides that esophageal IL-13 expression is associated with esophageal epithelial remodeling (BZH, DIS) and STAT3 protein expression, and that esophageal epithelial proliferation is associated with increased STAT3.

### IL-13-Induced Esophageal Epithelial Proliferation is STAT3-Dependent.

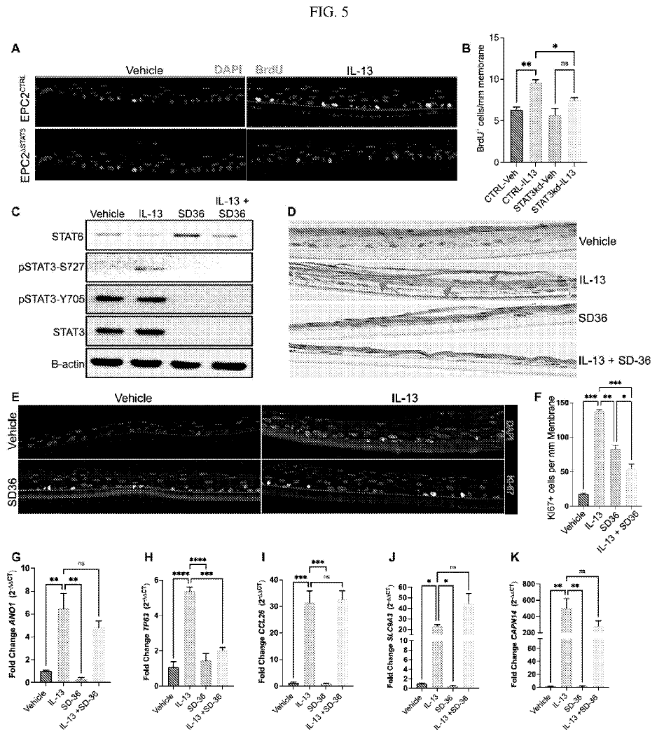

In order to determine the requirement of STAT3 in esophageal epithelial proliferation, BrdU incorporation in IL-13-stimulated EPC2-ALI with shRNA-mediated knockdown of STAT3 (EPC2-ALIASTAT3) or empty-vector shRNA (EPC2-ALICTRL) was examined. Under steady state conditions no difference in BrdU incorporation in EPC2-ALICTRL and EPC2-ALIASTAT3 was observed, however IL-13-stimulation induced a significant increase in BrdU incorporation in EPC2-ALICTRL cells (FIG. 5A-5B). In contrast, IL-13-induced proliferation was abrogated in EPC2-ALIASTAT3 cells indicating that IL-13-induced proliferation was STAT3-dependent (FIG. 5A-5B). To determine whether this was a consequence of developmental loss of STAT3 in EPC2 cell functionality, the STAT3 protein degrader, SD-36 (10 uM)45 was used. SD-36 is a potent and selective PROTAC (proteolysis-targeting chimera) degrader which serves as an endogenous protein degradation tool. SD-36 serves as a “linker” molecule which tethers STAT3 to an E3 ligase for ubiquitin-mediated proteasomal degradation45. Treatment of EPC2-ALI cells with SD-36 led to degradation of STAT3 at steady state and following IL-13-stimulation (100 ng/mL) (FIG. 5 C). Importantly SD36 degradation was associated with total loss of IL-13 induced phosphorylation of STAT3 Y705 and Y727 residues (FIG. 5C). IL-13 exposure of EPC2-ALI leads to epithelial remodeling including basal cell proliferation (BZH), as evidenced by Ki-67+ staining and DIS) (FIG. 5D-5F; BZH indicated by yellow bracket, DIS indicated by green arrows). Consistent with the genetic approach, the IL-13-induced increase in EPC2-ALI Ki-67+ cells were attenuated following STAT3 degradation (FIG. 5F). To determine the effect of loss of STAT3 on IL-13-induction of key EoE genes RT-qPCR was performed for mRNA expression of surrogate EoE proliferation genes, ANO1 and TP63, and canonical EoE inflammatory genes, CCL26, SLC9A3, and CAPN14, in the presence and absence of SD36. IL-13 significantly induced mRNA expression of ANO1 (FIG. 5G) and TP63 (FIG. 5H), and this effect was abolished in the presence of SD-36. Surprisingly, IL-13 induction of canonical EoE inflammatory genes was not significantly inhibited by STAT3 degradation (FIG. 5I-5K). Thus, the disclosure provides that STAT3 is required specifically for IL-13-induced esophageal epithelial proliferation and expression of key proliferation genes and not for IL-13-induced canonical inflammation genes (i.e., CCL26 and CAPN14).

SFRP1 is a Novel Candidate Gene which is Associated with Cell Proliferation and is a STAT3 Target.

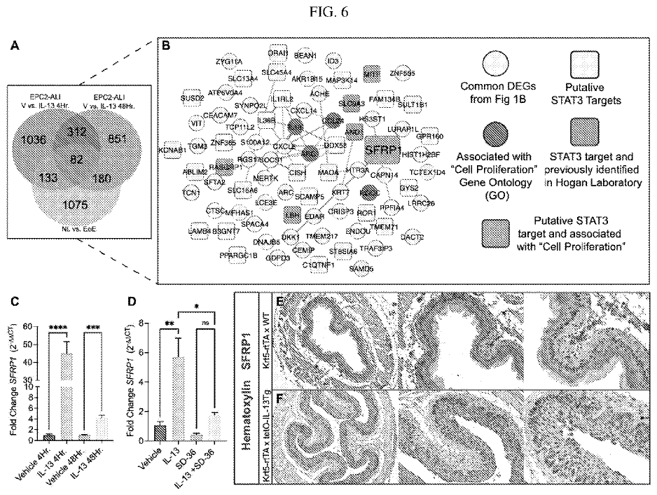

To gain a mechanistic understanding for how IL-13-induced STAT3-dependent signaling drives esophageal epithelial proliferation, advanced in silico analysis was performed on the 82 DEGs identified from the RNAseq analyses in FIG. 1A (FIG. 6A). The 32 putative STAT3 targets identified from n=82 common DEGs were mapped onto the “Cell Proliferation” Gene Ontology (GO:0008283; n=2148 genes) and identified n=4 genes, Secreted Frizzled-Related Protein-1 (SFRP1), Melanocyte-inducing Transcription Factor (MITF), LBH Regulator of WNT Signaling Pathway (LBH), and RAS Guanyl Releasing Protein-1 (RASGRP1) (FIG. 6B). Given the identified interaction between SFRP1 and the key EoE gene, CAPN14 and that SFRP1 plays a critical role for in the regulation of canonical WNT signaling and cell proliferation, the role of SFRP1 in IL-13-induced STAT3-dependent esophageal epithelial proliferation was examined. Notably, IL-13-stimulation of EPC2-ALI induced a significant increase in SFRP1 mRNA by 4 hours which returned to basal levels by 48 hours (FIG. 6C), and that IL-13-induction of SFRP1 mRNA was STAT3-dependent (FIG. 6D). The disclosure therefore provides a functional role for SFRP1 in regulating IL-13-induced STAT3-dependent esophageal epithelial proliferation.

### Esophageal Epithelial Cells are Cellular Sources of SFRP1 Expression and IL-13-Induced Esophageal Proliferation is Regulated by SFRP1.

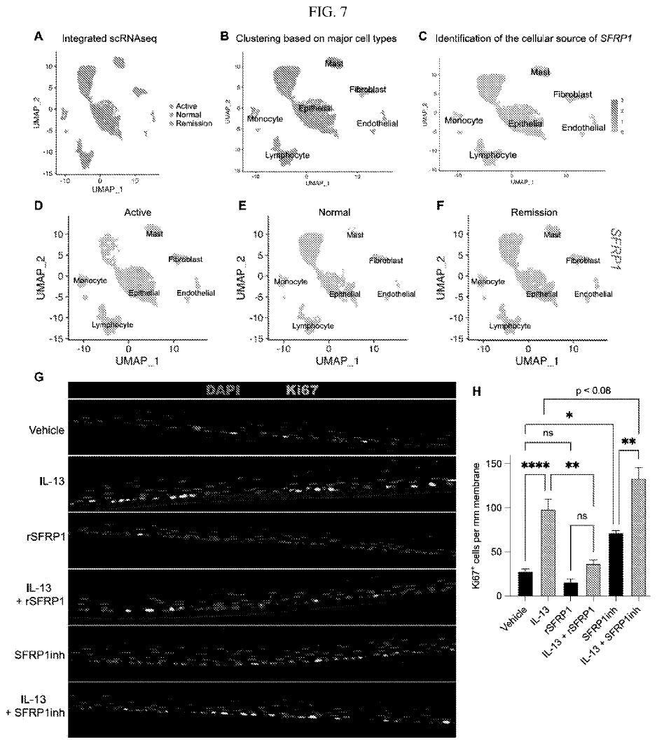

To determine the predominant cellular sources of SFRP1, publicly available single-cell RNAseq data of esophageal biopsies were used from healthy control, disease remission, and active disease patients (GSE201153; n=2 healthy control, n=3 remission, n=5 active disease)46. It was identified that there was increased SFRP1 mRNA expression in biopsies of patients with active disease, specifically within the epithelial cluster, compared to biopsies of patients in remission or healthy controls (FIG. 7A-7F). To define the role of SFRP1 in esophageal epithelial proliferation, IL-13 stimulation of EPC2-ALI cells in presence of a pharmacologic agonist (r-SFRP1) and antagonist of SFRP1 (SFRP1inh) was characterized. IL-13-stimulation of EPC2-ALI cells induced proliferation (FIG. 7G-7H). Il-13-stimulation of EPC2-ALI cells in the presence of rSFRP1 resulted in a decrease in Ki67+ EPC2-ALI cells (FIG. 7G-7H). In contrast, IL-13-stimulation of EPC2-ALI in the presence of SFRP1inh resulted in an increase in Ki67+ cells compared to IL-13 alone suggesting that SFRP1 blockade enhances IL-13-induced esophageal epithelial proliferation. (FIG. 7G-7H). Intriguingly, treatment of EPC2-ALI cells with rSFRP1 alone was sufficient to inhibit esophageal epithelial proliferation; conversely, treatment of EPC2-ALI with SFRP1inh resulted in an increase in Ki67+ cells compared to steady-state (Vehicle) (FIG. 7G-7H). The disclosure provides that SFRP1 is predominantly produced in esophageal epithelial cells in active disease, and that SFRP1 regulations steady state and IL-13-induced esophageal epithelial proliferation in vitro.

A Novel Esophageal Epithelial Cell Cluster Present Exclusively in Active EoE scRNAseq Samples is a Potent Source of SFRP1.

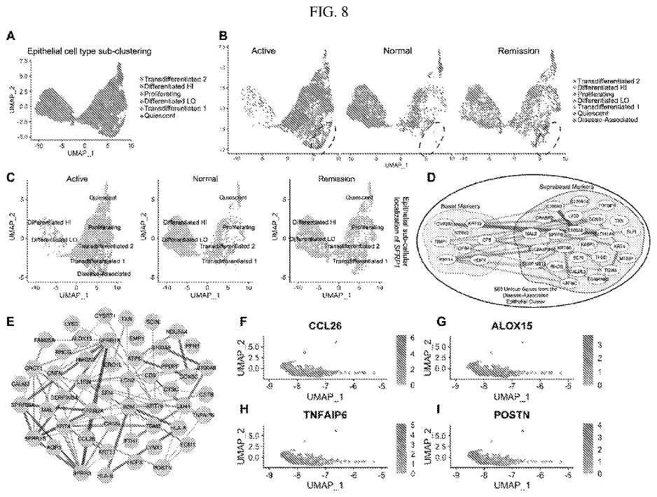

Given that SFRP1 expression was predominantly restricted to the esophageal epithelial cluster of individuals with active disease (FIG. 7), the epithelial cells were further sub-clustered in order to determine the subset of epithelial cells that are the major source of SFRP1 and the relationship with esophageal epithelial proliferation. Sub-clustering of the epithelial cells was also utilized in order to provide insight into the SFRP1+ esophageal epithelial cell phenotype and proliferative signaling pathways that were coexpressed with SFRP1. Subclustering of the epithelial populations within the 10 integrated samples derived from the 3 disease states (healthy, remission, and active disease) led to the generation of 13 communities that were categorized into 6 main epithelial clusters, defined by Rochman et al. (Rochman et al., JCI Insights, 2022:7e159093), including “Quiescent,” “Proliferating,” “Transdifferentiated 1,” “Transdifferentiated 2,” “Differentiated HI,” and “Differentiated LO” (See FIGS. 8A and 8B). Stratification of the integrated data into the 3 disease states (healthy, remission, and active disease) revealed a seventh epithelial subcluster that was restricted to the active disease (FIGS. 8A and 8B). Mapping SFRP1 expression on the subclustered epithelial populations identified that SFRP1 expression was predominantly restricted to the seventh epithelial population that was unique to the active disease group (FIG. 8C). Differential gene expression analyses of the active disease-specific SFRP1+ cell population identified 120 DEGs. This seventh epithelial subcluster comprised a suprabasal epithelial cell signature (8 basal markers and 25 suprabasal markers; FIG. 8D). Intriguingly, the SFRP1+ suprabasal epithelial cell population highly differentially expressed the canonical EoE disease-driving inflammatory genes such as TNFAIP6, POSTN, CCL26, and ALOX15 as compared with the 6 other epithelial clusters (FIGS. 8E, 8F, 8G, 8H, and 8I) (Sherrill et al., Genes Immno 2014; 15:361-369). Thus, the disclosure identified SFRP1+ disease-associated esophageal epithelial cells as a suprabasal epithelial population that expressed the EoE inflammatory signature. Analysis of this epithelial cell population identified a variety of markers useful for identification of epithelial disease cells, for example CD44, CD9, and HLA-B. Thus, the disclosure provides that SFRP1+ esophageal epithelial cells are a suprabasal epithelial cell population (e.g., characterized by markers identified in FIG. 8E (e.g., expression of cell surface proteins CD44, CD9 and HLAB), and/or expression of one or more inflammatory genes such as TNFAIP6, POSTN, CCL26, and ALOX15) that underlie (e.g., cause and/or drive) EoE-inflammatory disease (e.g., that can be used as markers for the disease and/or targeted for treatment of disease).

It is to be understood that the foregoing detailed description and accompanying examples are merely illustrative and are not to be taken as limitations upon the scope of the disclosure. Various changes and modifications to the disclosed embodiments will be apparent to those skilled in the art. Such changes and modifications, including without limitation those relating to the biological agents, structures, substituents, derivatives, intermediates, syntheses, compositions, formulations, or methods of use of the disclosure, may be made without departing from the spirit and scope thereof. Thus, it is intended that the present disclosure cover modifications and variations thereof provided they come within the scope of the appended claims and/or their equivalents.

### REFERENCES

1. Aceves S S, Ackerman S J. Relationships between eosinophilic inflammation, tissue remodeling, and fibrosis in eosinophilic esophagitis. Immunol Allergy Clin North Am 2009; 29:197-211, xiii-xiv.
2. Collins M H. Histopathology of eosinophilic esophagitis. Dig Dis 2014; 32:68-73.
3. Dellon E S. Cost-effective care in eosinophilic esophagitis. Ann Allergy Asthma Immunol 2019; 123:166-72.
4. Furuta G T, Katzka D A. Eosinophilic Esophagitis. N Engl J Med 2015; 373:1640-8.
5. Gonsalves N P, Aceves S S. Diagnosis and treatment of eosinophilic esophagitis. Journal of Allergy and Clinical Immunology 2020; 145:1-7.
6. Warners M J, van Rhijn B D, Verheij J, Smout A, Bredenoord A J. Disease activity in eosinophilic esophagitis is associated with impaired esophageal barrier integrity. Am J Physiol Gastrointest Liver Physiol 2017; 313:G230-g8.
7. Blanchard C, Mingler M K, Vicario M, Abonia J P, Wu Y Y, Lu T X, et al. IL-13 involvement in eosinophilic esophagitis: transcriptome analysis and reversibility with glucocorticoids. J Allergy Clin Immunol 2007; 120:1292-300.
8. Cheng E, Souza R F, Spechler S J. Tissue remodeling in eosinophilic esophagitis. Am J Physiol Gastrointest Liver Physiol 2012; 303:G1175-87.
9. Vanoni S, Zeng C, Marella S, Uddin J, Wu D, Arora K, et al. Identification of anoctamin I (ANO1) as a key driver of esophageal epithelial proliferation in eosinophilic esophagitis. J Allergy Clin Immunol 2020; 145:239-54 e2.
10. Aceves S S. Remodeling and fibrosis in chronic eosinophil inflammation. Dig Dis 2014; 32:15-21.
11. Muir A B, Wang J X, Nakagawa H. Epithelial-stromal crosstalk and fibrosis in eosinophilic esophagitis. J Gastroenterol 2019; 54:10-8.
12. Mulder D J, Justinich C J. Understanding eosinophilic esophagitis: the cellular and molecular mechanisms of an emerging disease. Mucosal Immunol 2011; 4:139-47.
13. Warners M J, Vlieg-Boerstra B J, Verheij J, van Hamersveld PHP, van Rhijn B D, Van Ampting M T J, et al. Esophageal and Small Intestinal Mucosal Integrity in Eosinophilic Esophagitis and Response to an Elemental Diet. Am J Gastroenterol 2017; 112:1061-71.
14. Akei H S, Mishra A, Blanchard C, Rothenberg M E. Epicutaneous Antigen Exposure Primes for Experimental Eosinophilic Esophagitis in Mice. Gastroenterology 2005; 129:985-94.
15. Collins M H. Histopathologic features of eosinophilic esophagitis. Gastrointest Endosc Clin N Am 2008; 18:59-71; viii-ix.
16. Doherty T A, Baum R, Newbury R O, Yang T, Dohil R, Aquino M, et al. Group 2 innate lymphocytes (ILC2) are enriched in active eosinophilic esophagitis. J Allergy Clin Immunol 2015; 136:792-4.e3.
17. Bullock J Z, Villanueva J M, Blanchard C, Filipovich A H, Putnam P E, Collins M H, et al. Interplay of adaptive th2 immunity with eotaxin-3/c-C chemokine receptor 3 in eosinophilic esophagitis. J Pediatr Gastroenterol Nutr 2007; 45:22-31.
18. Cianferoni A, Ruffner M A, Guzek R, Guan S, Brown-Whitehorn T, Muir A, et al. Elevated expression of activated T(H)2 cells and milk-specific T(H)2 cells in milk-induced eosinophilic esophagitis. Ann Allergy Asthma Immunol 2018; 120:177-83.e2.
19. Wen T, Aronow B J, Rochman Y, Rochman M, Ke K, Dexheimer P J, et al. Single-cell RNA sequencing identifies inflammatory tissue T cells in eosinophilic esophagitis. J Clin Invest 2019; 129:2014-28.
20. D'Mello R J, Caldwell J M, Azouz N P, Wen T, Sherrill J D, Hogan S P, et al. LRRC31 is induced by IL-13 and regulates kallikrein expression and barrier function in the esophageal epithelium. Mucosal Immunol 2016; 9:744-56.
21. Zuo L, Fulkerson P C, Finkelman F D, Mingler M, Fischetti C A, Blanchard C, et al. IL-13 induces esophageal remodeling and gene expression by an eosinophil-independent, IL-13R alpha 2-inhibited pathway. J Immunol 2010; 185:660-9.
22. Hirano I, Collins M H, Assouline-Dayan Y, Evans L, Gupta S, Schoepfer A M, et al. RPC4046, a Monoclonal Antibody Against IL13, Reduces Histologic and Endoscopic Activity in Patients With Eosinophilic Esophagitis. Gastroenterology 2019; 156:592-603.e10.
23. Dellon E S, Collins M H, Rothenberg M E, Assouline-Dayan Y, Evans L, Gupta S, et al. Long-term Efficacy and Tolerability of RPC4046 in an Open-Label Extension Trial of Patients With Eosinophilic Esophagitis. Clin Gastroenterol Hepatol 2021; 19:473-83.e17.
24. Hirano I, Dellon E S, Hamilton J D, Collins M H, Peterson K, Chehade M, et al. Efficacy of Dupilumab in a Phase 2 Randomized Trial of Adults With Active Eosinophilic Esophagitis. Gastroenterology 2020; 158:111-22.e10.
25. Dellon E S, Rothenberg M E, Collins M H, Hirano I, Chehade M, Bredenoord A J, et al. A Phase 3, Randomized, 3-Part Study to Investigate the Efficacy and Safety of Dupilumab in Adult and Adolescent Patients With Eosinophilic Esophagitis: Results From Part A. American College of Gastroenterology 2020 Annual Meeting. Virtual, 2020.
26. Sherrill J D, Kiran K C, Blanchard C, Stucke E M, Kemme K A, Collins M H, et al. Analysis and expansion of the eosinophilic esophagitis transcriptome by RNA sequencing. Genes Immun 2014; 15:361-9.
27. Bhattacharjee A, Shukla M, Yakubenko V P, Mulya A, Kundu S, Cathcart M K. IL-4 and IL-13 employ discrete signaling pathways for target gene expression in alternatively activated monocytes/macrophages. Free Radic Biol Med 2013; 54:1-16.
28. Gann P H, Deaton R J, McMahon N, Collins M H, Dellon E S, Hirano I, et al. An anti-IL-13 antibody reverses epithelial-mesenchymal transition biomarkers in eosinophilic esophagitis: Phase 2 trial results. J Allergy Clin Immunol 2020; 146:367-76 e3.
29. Gour N, Wills-Karp M. IL-4 and IL-13 signaling in allergic airway disease. Cytokine 2015; 75:68-78.
30. Rothenberg M E, Wen T, Greenberg A, Alpan O, Enav B, Hirano I, et al. Intravenous anti-IL-13 mAb QAX576 for the treatment of eosinophilic esophagitis. J Allergy Clin Immunol 2015; 135:500-7.
31. Roy S, Liu H Y, Jaeson M I, Deimel L P, Ranasinghe C. Unique IL-13Ralpha2/STAT3 mediated IL-13 regulation detected in lung conventional dendritic cells, 24 h post viral vector vaccination. Sci Rep 2020; 10:1017.
32. Schmidt H, Braubach P, Schilpp C, Lochbaum R, Neuland K, Thompson K, et al. IL-13 Impairs Tight Junctions in Airway Epithelia. Int J Mol Sci 2019; 20.
33. Miller D E, Forney C, Rochman M, Cranert S, Habel J, Rymer J, et al. Genetic, Inflammatory, and Epithelial Cell Differentiation Factors Control Expression of Human Calpain-14. G3 (Bethesda) 2019; 9:729-36.
34. Mishra A, Rothenberg M E. Intratracheal IL-13 induces eosinophilic esophagitis by an IL-5, eotaxin-1, and STAT6-dependent mechanism. Gastroenterology 2003; 125:1419-27.
35. Miloux B, Laurent P, Bonnin O, Lupker J, Caput D, Vita N, et al. Cloning of the human IL-13R alpha1 chain and reconstitution with the IL4R alpha of a functional IL-4/IL-13 receptor complex. FEBS Lett 1997; 401:163-6.
36. Schnyder B, Lugli S, Feng N, Etter H, Lutz R A, Ryffel B, et al. Interleukin-4 (IL-4) and IL-13 bind to a shared heterodimeric complex on endothelial cells mediating vascular cell adhesion molecule-1 induction in the absence of the common gamma chain. Blood 1996; 87:4286-95.
37. Johnston P A, Grandis J R. STAT3 signaling: anticancer strategies and challenges. Mol Interv 2011; 11:18-26.
38. Umeshita-Suyama R, Sugimoto R, Akaiwa M, Arima K, Yu B, Wada M, et al. Characterization of IL-4 and IL-13 signals dependent on the human IL-13 receptor alpha chain 1: redundancy of requirement of tyrosine residue for STAT3 activation. Int Immunol 2000; 12:1499-509.
39. Dellon E S, Liacouras C A, Molina-Infante J, Furuta G T, Spergel J M, Zevit N, et al. Updated International Consensus Diagnostic Criteria for Eosinophilic Esophagitis: Proceedings of the AGREE Conference. Gastroenterology 2018; 155:1022-33 e10.
40. Spergel J M, Dellon E S, Liacouras C A, Hirano I, Molina-Infante J, Bredenoord A J, et al. Summary of the updated international consensus diagnostic criteria for eosinophilic esophagitis: AGREE conference. Ann Allergy Asthma Immunol 2018; 121:281-4.
41. Zeng C, Vanoni S, Wu D, Caldwell J M, Wheeler J C, Arora K, et al. Solute carrier family 9, subfamily A, member 3 (SLC9A3)/sodium-hydrogen exchanger member 3 (NHE3) dysregulation and dilated intercellular spaces in patients with eosinophilic esophagitis. J Allergy Clin Immunol 2018; 142:1843-55.
42. Lu T X, Sherrill J D, Wen T, Plassard A J, Besse J A, Abonia J P, et al. MicroRNA signature in patients with eosinophilic esophagitis, reversibility with glucocorticoids, and assessment as disease biomarkers. J Allergy Clin Immunol 2012; 129:1064-75 e9.
43. Ferrer-Torres D, Wu J H, Zhang C J, Hammer M A, Dame M K, Wu A, et al. Mapping the adult human esophagus in vivo and in vitro. Development 2022; 149.
44. Jiang M, Ku W Y, Zhou Z, Dellon E S, Falk G W, Nakagawa H, et al. BMP-driven NRF2 activation in esophageal basal cell differentiation and eosinophilic esophagitis. J Clin Invest 2015; 125:1557-68.
45. Bai L, Zhou H, Xu R, Zhao Y, Chinnaswamy K, McEachern D, et al. A Potent and Selective Small-Molecule Degrader of STAT3 Achieves Complete Tumor Regression In Vivo. Cancer Cell 2019; 36:498-511 e17.
46. Rochman M, Wen T, Kotliar M, Dexheimer P J, Ben-Baruch Morgenstern N, Caldwell J M, et al. Single-cell RNA-Seq of human esophageal epithelium in homeostasis and allergic inflammation. JCI Insight 2022; 7.
47. Sherrill J D, Kiran K, Blanchard C, Stucke E M, Kemme K A, Collins M H, et al. Analysis and expansion of the cosinophilic esophagitis transcriptome by RNA sequencing. Genes Immun 2014; 15:361-9.
48. Furue M. Regulation of Skin Barrier Function via Competition between AHR Axis versus IL-13/IL-4-JAK-STAT6/STAT3 Axis: Pathogenic and Therapeutic Implications in Atopic Dermatitis. J Clin Med 2020; 9.
49. Karpathiou G, Papoudou-Bai A, Ferrand E, Dumollard J M, Peoc'h M. STAT6: A review of a signaling pathway implicated in various diseases with a special emphasis in its usefulness in pathology. Pathol Res Pract 2021; 223:153477.
50. Hebenstreit D, Luft P, Schmiedlechner A, Duschl A, Horejs-Hoeck J. SOCS-1 and SOCS-3 inhibit IL-4 and IL-13 induced activation of Eotaxin-3/CCL26 gene expression in HEK293 cells. Mol Immunol 2005; 42:295-303.
51. Lv J, Xiong Y, Li W, Cui X, Cheng X, Leng Q, et al. IL-37 inhibits IL-4/IL-13-induced CCL11 production and lung eosinophilia in murine allergic asthma. Allergy 2018; 73:1642-52.
52. Hillmer E J, Zhang H, Li H S, Watowich S S. STAT3 signaling in immunity. Cytokine Growth Factor Rev 2016; 31:1-15.
53. Lee H, Jeong A J, Ye S K. Highlighted STAT3 as a potential drug target for cancer therapy. BMB Rep 2019; 52:415-23.
54. Dames P, Bergann T, Fromm A, Bucker R, Barmeyer C, Krug S M, et al. Interleukin-13 affects the epithelial sodium channel in the intestine by coordinated modulation of STAT6 and p38 MAPK activity. J Physiol 2015; 593:5269-82.
55. Muhl H. STAT3, a Key Parameter of Cytokine-Driven Tissue Protection during Sterile Inflammation—the Case of Experimental Acetaminophen (Paracetamol)-Induced Liver Damage. Front Immunol 2016; 7:163.
56. Kleuskens M T A, Haasnoot M L, Herpers B M, Ampting M, Bredenoord A J, Garssen J, et al. Butyrate and propionate restore interleukin 13-compromised esophageal epithelial barrier function. Allergy 2022; 77:1510-21.
57. Sherrill J D, Kc K, Wu D, Djukic Z, Caldwell J M, Stucke E M, et al. Desmoglein-1 regulates esophageal epithelial barrier function and immune responses in eosinophilic esophagitis. Mucosal Immunol 2014; 7:718-29.
58. Wheeler J C, Vanoni S, Zeng C, Waggoner L, Yang Y, Wu D, et al. 17beta-Estradiol protects the esophageal epithelium from IL-13-induced barrier dysfunction and remodeling. J Allergy Clin Immunol 2019; 143:2131-46.
59. Dong J, Cheng X D, Zhang W D, Qin J J. Recent Update on Development of Small-Molecule STAT3 Inhibitors for Cancer Therapy: From Phosphorylation Inhibition to Protein Degradation. J Med Chem 2021; 64:8884-915.
60. Lavergne E, Hendaoui I, Coulouarn C, Ribault C, Leseur J, Eliat P A, et al. Blocking Wnt signaling by SFRP-like molecules inhibits in vivo cell proliferation and tumor growth in cells carrying active beta-catenin. Oncogene 2011; 30:423-33.
61. Zhang H, Sun D, Qiu J, Yao L. SFRP1 inhibited the epithelial ovarian cancer through inhibiting Wnt/beta-catenin signaling. Acta Biochim Pol 2019; 66:393-400.
62. Sinnberg T, Makino E, Krueger M A, Velic A, Macek B, Rothbauer U, et al. A Nexus Consisting of Beta-Catenin and Stat3 Attenuates BRAF Inhibitor Efficacy and Mediates Acquired Resistance to Vemurafenib. EBioMedicine 2016; 8:132-49.
63. Baharudin R, Tieng F Y F, Lee L H, Ab Mutalib N S. Epigenetics of SFRP1: The Dual Roles in Human Cancers. Cancers (Basel) 2020; 12.
64. Wang Z, Li R, He Y, Huang S. Effects of secreted frizzled-related protein 1 on proliferation, migration, invasion, and apoptosis of colorectal cancer cells. Cancer Cell Int 2018; 18:48.
65. Liang C W, Yang C Y, Flavin R, Fletcher J A, Lu T P, Lai I R, et al. Loss of SFRP1 expression is a key progression event in gastrointestinal stromal tumor pathogenesis. Hum Pathol 2021; 107:69-79.
66. Bhattacharya B, Carlsten J, Sabo E, Kethu S, Meitner P, Tavares R, et al. Increased expression of eotaxin-3 distinguishes between eosinophilic esophagitis and gastroesophageal reflux disease. Hum Pathol 2007; 38:1744-53.
67. Matoso A, Mukkada V A, Lu S, Monahan R, Cleveland K, Noble L, et al. Expression microarray analysis identifies novel epithelial-derived protein markers in eosinophilic esophagitis. Mod Pathol 2013; 26:665-76.
68. Matoso A, Allen D, Herzlinger M, Ferreira J, Chen S, Lu S, et al. Correlation of ALOX15 expression with eosinophilic or reflux esophagitis in a cohort of pediatric patients with esophageal eosinophilia. Hum Pathol 2014; 45:1205-12.

1. A method of attenuating esophageal epithelial proliferation disease in a subject in need thereof, the method comprising increasing the level and/or the activity of secreted frizzled-related protein 1 (SFRP1) in the subject by administering to the subject an effective amount of:
a SFRP1 activator or a composition thereof; and/or
SFRP1, a biologically active fragment or variant thereof, or a nucleic acid encoding SFRP1 or a biologically active fragment or variant thereof.

2. The method of claim 1, wherein the activator comprises a small molecule activator of SFRP1.

3. The method of claim 1 or 2, wherein the activator comprises signal transducer and activator of transcription 3 (STAT3).

4. The method of any of claims 1-3, wherein the SFRP1 comprises an amino acid sequence having at least 70% identity to SEQ ID NO: 13.

5. The method of any of claims 1-4, wherein the SFRP1 comprises an amino acid sequence having at least 90% identity to SEQ ID NO: 13.

6. The method of any of claims 1-5, wherein the SFRP1 comprises an amino acid sequence of SEQ ID NO: 13.

7. The method of any of claims 1-6, wherein the esophageal epithelial proliferation disease is Basal Zone Hyperplasia (BZH).

8. The method of any of claims 1-6, wherein the esophageal epithelial proliferation disease is Eosinophilic Esophagitis (EoE)

9. A method of treating or preventing an epithelial cell disease or disorder in a subject comprising administering to the subject a therapeutically or prophylactically effective amount of a signal transducer and activator of transcription 3 (STAT3) inhibitor or a composition thereof to the subject.

10. The method of claim 9, wherein the disease or disorder is an esophageal epithelial proliferation disease.

11. The method of claim 10, wherein the esophageal epithelial proliferation disease is Basal Zone Hypeiplasia (BZH).

12. The method of claim 10, wherein the esophageal epithelial proliferation disease is Eosinophilic Esophagitis (EoE)

13. The method of any of claims 9-12, wherein the inhibitor is selected from the group consisting of a protein configured to bind STAT3 or a substrate thereof, a gene silencing oligonucleotide, a small molecule inhibitor of STAT3, a negative allosteric modulating agent, a protease, and combinations thereof.

14. The method of any of claims 9-13, wherein the STAT3 inhibitor is a peptide inhibitor.

15. The method of claim 14, wherein the peptide inhibitor is selected from DBD-1, ISS-610, and PY*LKTK.

16. The method of any of claims 9-13, wherein the STAT3 inhibitor is a small molecule inhibitor.

17. The method of claim 16, wherein the small molecule inhibitor is selected from PLLL32, HJC0152, LL1, LLL-3, LLL12, LYW-6, nitidine chloride, SD-36, static, STX-0119, S31-1757, S31-201, CPA-7, C48, GPA512, and MMPP.

18. The method of any of claims 9-13, wherein the STAT3 inhibitor is an oligonucleodite.

19. The method of claim 18, wherein the nucleotide is InS3-54A18 or STAT3hpdODN.

20. A method of treating or preventing a disease or disorder in a subject comprising administering to the subject a therapeutically or prophylactically effective amount of at least one signal transducer and activator of transcription 3 (STAT3) inhibitor or a composition thereof,
wherein the disease or disorder is characterized by aberrant esophageal epithelial proliferation, and
wherein the epithelial proliferation occurs within a suprabasal epithelial cell population of cells expressing one or more inflammatory genes selected from TNFAIP6, POSTN, CCL26, and ALOX15.

21. The method of claim 20, wherein the suprabasal epithelial cell population of cells express the inflammatory genes TNFAIP6, POSTN, CCL26, and ALOX15.

22. The method of claim 20 or claim 21, wherein administration of the at least one STAT3 inhibitor results in at least one of the following in the subject: attenuation of aberrant esophageal epithelial proliferation, a decrease in epithelial cell remodeling, a reduction in inflammation, or a combination thereof.

23. The method of any of claims 20-22, wherein the disease or disorder is Basal Zone Hyperplasia (BZH).

24. The method of any of claims 20-22, wherein the disease or disorder is Eosinophilic Esophagitis (EoE).

25. The method of any of claims 20-24, wherein the inhibitor is selected from the group consisting of a protein configured to bind STAT3 or a substrate thereof, a gene silencing oligonucleotide, a small molecule inhibitor of STAT3, a negative allosteric modulating agent, a protease, and combinations thereof.

26. The method of any of claims 20-25, wherein the STAT3 inhibitor is peptide inhibitor.

27. The method of claim 26, wherein the peptide inhibitor is selected from DBD-1, ISS-610, and PY*LKTK.

28. The method of any of claims 20-25, wherein the STAT3 inhibitor is a small molecule inhibitor.

29. The method of claim 28, wherein the small molecule inhibitor is selected from PLLL32, HJC0152, LL1, LLL-3, LLL12, LYW-6, nitidine chloride, SD-36, static, STX-0119, S31-1757, S31-201, CPA-7, C48, GPA512, MMPP.

30. The method of any of claims 20-25, wherein the STAT3 inhibitor is an oligonucleodite.

31. The method of claim 30, wherein the nucleotide is InS3-54A18 or STAT3hpdODN.

32. The method of any of claims 20-25, further comprising administration of at least one additional therapeutic agent.

33. The method of claim 32, wherein the at least one additional therapeutic agent comprises a chemotherapeutic, a corticosteroid, an immunosuppressant, an anti-inflammatory agent, an antibiotic, or any combination thereof.

34. The method of any of claims 1-33, wherein the administration is via a route selected from intravenously, arterially, intradermally, intramuscularly, intraperitoneally, subcutaneously, ocularly, sublingually, orally, intranasally, intraspinally, intracerebrally, and transdermally.

35. A method of treating or preventing a disease or disorder in a subject, comprising administering to the subject an effective amount of secreted frizzled-related protein 1 (SFRP1), a biologically active fragment or variant thereof, or a nucleic acid encoding SFRP1 or a biologically active fragment or variant thereof.

36. The method of claim 35, wherein the disease or disorder is characterized by esophageal epithelial proliferation.

37. The method of claim 35 or 36, wherein the disease or disorder is Basal Zone Hyperplasia (BZH).

38. The method of claim 35 or 36, wherein the disease or disorder is Eosinophilic Esophagitis (EoE).

39. The method of any of claims 35-38, wherein the subject has or is suspected of having an esophageal epithelial remodeling disorder.

40. The method of any of claims 35-39, wherein the SFRP1 comprises an amino acid sequence having at least 70% identity to SEQ ID NO: 1.

41. The method of any of claims 35-39, wherein the SFRP1 comprises an amino acid sequence having at least 90% identity to SEQ ID NO: 1.

42. The method of any of claims 35-39, wherein the SFRP1 comprises an amino acid sequence of SEQ ID NO: 1.

43. The method of any of claims 35-39, further comprising administering a signal transducer and activator of transcription 3 (STAT3) inhibitor or a composition thereof.

44. Use of a SFRP1 activator or a composition thereof; and/or SFRP1, a biologically active fragment or variant thereof, or a nucleic acid encoding SFRP1 or a biologically active fragment or variant thereof, in treating a disease or disorder characterized by esophageal epithelial proliferation.

45. Use of a SFRP1 activator or a composition thereof; and/or SFRP1, a biologically active fragment or variant thereof, or a nucleic acid encoding SFRP1 or a biologically active fragment or variant thereof, in treating a disease or disorder characterized by esophageal epithelial remodeling.

46. Use of a SFRP1 activator or a composition thereof; and/or SFRP1, a biologically active fragment or variant thereof, or a nucleic acid encoding SFRP1 or a biologically active fragment or variant thereof, in regulating esophageal epithelial proliferation.

47. Use of a signal transducer and activator of transcription 3 (STAT3) inhibitor or a composition thereof for treating or preventing a disease or disorder in a subject comprising administering to the subject a therapeutically or prophylactically effective amount of a STAT3 inhibitor or a composition thereof to the subject, wherein the disease or disorder is an esophageal epithelial proliferation disease.

48. The use according to claim 47, wherein the esophageal epithelial proliferation disease is Basal Zone Hyperplasia (BZH) or Eosinophilic Esophagitis (EoE).


SEQUENCE LISTING (13 sequences)

SEQ ID NO: 1 - protein, 20 aa, synthetic construct
  REGION 1..20: note = Synthetic
CTCAACAAGAACTGCCACGC

SEQ ID NO: 2 - protein, 19 aa, synthetic construct
  REGION 1..19: note = Synthetic
CTCGTTGTCACAGGGAGGA

SEQ ID NO: 3 - protein, 22 aa, synthetic construct
  REGION 1..22: note = Synthetic
GAGCCAAAGACATCGGAATCTG

SEQ ID NO: 4 - protein, 21 aa, synthetic construct
  REGION 1..21: note = Synthetic
TGAAGGAGATCACGAAGGCAT

SEQ ID NO: 5 - protein, 18 aa, synthetic construct
  REGION 1..18: note = Synthetic
AAGCGCCCGTTTCGTCAG

SEQ ID NO: 6 - protein, 20 aa, synthetic construct
  REGION 1..20: note = Synthetic
TGTGCTGAGGAAGGTACTGC

SEQ ID NO: 7 - protein, 20 aa, synthetic construct
  REGION 1..20: note = Synthetic
AGTGCTGCTTCTGTTCCCAA

SEQ ID NO: 8 - protein, 20 aa, synthetic construct
  REGION 1..20: note = Synthetic
AAGGTGGAGACTCAGGAGGG

SEQ ID NO: 9 - protein, 22 aa, synthetic construct
  REGION 1..22: note = Synthetic
CAATGACGGAGAATTCTGGATG

SEQ ID NO: 10 - protein, 22 aa, synthetic construct
  REGION 1..22: note = Synthetic
GTTCTTCCAAAATGTGTCCTGC

SEQ ID NO: 11 - protein, 23 aa, synthetic construct
  REGION 1..23: note = Synthetic
CAGACTGAAGAGCTATTGTAATG

SEQ ID NO: 12 - protein, 23 aa, synthetic construct
  REGION 1..23: note = Synthetic
CCAGTGTCAATTATATCTTCCAC

SEQ ID NO: 13 - protein, 314 aa, synthetic construct
  REGION 1..314: note = Synthetic
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK